(19) United States
(12) Patent Application Publication
(10) Pub. No.: US 2020/0176118 A1
(43) Pub. Date: Jun. 4, 2020

(54) METHODS AND SYSTEMS FOR SEPTIC SHOCK RISK ASSESSMENT

(71) Applicant: Berg LLC, Framingham, MA (US)

(72) Inventors: Vijetha Vemulapalli, Westborough, MA (US); Niven Rajin Narain, Cambridge, MA (US); Viatcheslav R. Akmaev, Sudbury, MA (US)

(21) Appl. No.: 16/673,885

(22) Filed: Nov. 4, 2019

Related U.S. Application Data
(60) Provisional application No. 62/831,102, filed on Apr. 8, 2019.
(60) Provisional application No. 62/755,556, filed on Nov. 4, 2018.

(51) Int. Cl. G16H 50/30; G16H 10/60; G16H 40/63; G16H 40/67; A61B 5/145; A61B 5/00

(52) U.S. Cl. CPC G16H 50/30; G16H 10/60; G16H 40/63; A61B 2505/03; A61B 5/14532; A61B 5/14539; A61B 5/412; G16H 40/67

(57) ABSTRACT

Provided herein are methods and systems for determining the risk of a septic shock in a patient or for identifying a patient at high risk if septic shock. In some embodiment a method includes accessing and/or receiving information regarding a patient, the information including an indication of whether a measured level of mean corpuscular hemoglobin for the patient fell outside a normal range for the patient, and an indication of whether a measured level of whole blood potassium for the patient fell outside a normal range for the patient. In some embodiments, the method includes determining an estimated risk of the patient experiencing septic shock within a specified time period based on, at least, the indication of whether the measured level of mean corpuscular hemoglobin fell outside the normal range and the indication of whether the measured level of whole blood potassium fell outside the normal range.

### RELATED APPLICATION

This application claims priority to and benefit of U.S. Provisional Patent Application No. 62/755,556, filed Nov. 4, 2018, and U.S. Provisional Patent Application No. 62/831,102, filed Apr. 8, 2019, the entire contents of each of which are expressly incorporated herein by reference.

### BACKGROUND

Sepsis is a serious and life threatening condition that arises when the body's response to infection causes injury to its own tissues and organs and is a great cause of concern among hospitalized patients. In sepsis, a systemic inflammatory response (SIRS) is caused by the infection. Sepsis can progress into severe sepsis accompanied by remote organ dysfunction. Septic shock, which is characterized by severe sepsis accompanied by low blood pressure and no response to fluid replacement, is the most severe form of sepsis that involves presence of arterial hypotension and is associated with significantly worse outcomes with mortality rates estimated to be as high as 40% (see G. S. Martin, “Sepsis, severe sepsis and septic shock: changes in incidence, pathogens and outcomes,” Expert Rev Anti Infect Ther, vol. 10, no. 6, pp. 701-706, June 2012). The incidence rate of sepsis was found to be around 6% in 2014 with about 15% of those patients dying as well as 6.2% of those being admitted to hospice care (see C. Rhee et al., “Incidence and Trends of Sepsis in US Hospitals Using Clinical vs Claims Data, 2009-2014,” JAMA, vol. 318, no. 13, pp. 1241-1249, October 2017). In addition to the high incidence of sepsis, it is among the most expensive illnesses to treat in the US costing a median of around $32,421 per patient for the entire hospital stay as well as $27,461 for the Intensive Care Unit (ICU) costs (see Arefian et al., “Hospital-related cost of sepsis: A systematic review,” Journal of Infection, vol. 74, no. 2, pp. 107-117, February 2017).

As of now, there is no gold standard for diagnosis and prediction of sepsis and septic shock. Septic shock is currently diagnosed based on a combination of lab tests and clinical features such as fever, low blood pressure and difficulty breathing. Early diagnosis of this condition is complicated by non-specific clinical signs and symptoms and the fact that not all infections lead to sepsis and to progression into septic shock. The current standard of care for septic shock includes administration of antibiotics, antifungal drugs, regulating blood volume and ensuring sufficient tissue perfusion. Surgical source control is a measure that is used less often and is recommended at the earliest possible time to obtain the best outcome. Early detection of sepsis and early administration of antibiotic treatment has been known to be one of the strongest modulators of outcomes in patients with sepsis. While highly desirable, early diagnosis is also more challenging to accomplish in a clinical setting as discussed earlier. Given the non-specific nature of early symptoms, it is impractical to closely monitor all patients in the ICU. Early diagnosis of sepsis and septic shock has been unambiguously linked to lower mortality and better patient outcomes. Early prediction of development of sepsis is highly valuable because it provides an opportunity for intervention and treatment that improves patient outcomes and thereby also eliminates associated healthcare costs.

Accordingly, improved methods and systems are needed for early prediction of development of sepsis.

### SUMMARY

Embodiments described herein provide methods and systems for determination risk of a patient developing sepsis. Some embodiments provide methods and systems for determination of a risk of a patient developing sepsis based on measured levels of mean corpuscular hemoglobin and whole blood potassium being outside a normal range for a patient. In some embodiments, the normal range is based on patient specific factors.

An embodiment of the invention provides a method that includes accessing and/or receiving information regarding a patient, the information including an indication of whether a measured level of mean corpuscular hemoglobin for the patient fell outside a normal range for the patient, and an indication of whether a measured level of whole blood potassium for the patient fell outside a normal range for the patient. One or more microcontrollers can determine an estimated risk of the patient experiencing septic shock within a specified time period based on, at least, the indication of whether the measured level of mean corpuscular hemoglobin fell outside the normal range and the indication of whether the measured level of whole blood potassium fell outside the normal range. In some embodiments, the method includes providing information regarding the estimated risk of the patient experiencing septic shock within the specified time period.

In some embodiments, the accessed and/or received information regarding the patient includes information regarding the whether the patient has been diagnosed with at least one disease or disorder in a group of diseases and disorders, and determination of the estimated risk of the patient experiencing septic shock within the specified time period is based on, at least, the indication of whether the measured level of mean corpuscular hemoglobin fell outside the normal range, the indication of whether the measured level of whole blood potassium fell outside the normal range, and the information regarding whether the patient has been diagnosed with at least one disease or disorder in the group.

In some embodiments, determining of the estimated risk of the patient experiencing septic shock within the specified time period is based on the indication of whether the measured level of mean corpuscular hemoglobin fell outside the normal range, the indication of whether the measured level of whole blood potassium fell outside the normal range, and the information regarding whether the patient has been diagnosed with at least one disease or disorder in the group.

In some embodiments, determining the estimated risk of the patient experiencing septic shock within the specified time period is based, at least in part, on a statistical model of risk using a first variable based on the indication of whether the measured level of mean corpuscular hemoglobin fell outside the normal range, a second variable based on the indication of whether the measured level of whole blood potassium fell outside the normal range, and a third variable based on whether the patient has been diagnosed with at least one disease or disorder in the group.

In some embodiments, determining the estimated risk of the patient experiencing septic shock within the specified time period is based on a statistical model of risk using a first variable based on the indication of whether the measured level of mean corpuscular hemoglobin fell outside the normal range, a second variable based on the indication of whether the measured level of whole blood potassium fell outside the normal range, and a third variable based on whether the patient has been diagnosed with at least one disease or disorder in the group.

In some embodiments the model is a statistical regression model based on the first variable, the second variable, and the third variable.

In some embodiments, a value of the first variable is zero or non-zero based on the indication of whether the measured level of mean corpuscular hemoglobin fell outside the normal range. For example, the first variable is zero if the measured level fell in the normal range and is nonzero if the measured level fell outside the normal range. A value of the second variable is zero or non-zero based on the indication of whether the measured level of whole blood potassium fell outside the normal rage. For example, the second variable is zero if the measured level fell in the normal range and is nonzero if the measured level fell outside the normal range. A value of the third variable is zero or non-zero based on whether the patient has been diagnosed with at least one disease or disorder in the group. For example, the value of the third variable is nonzero if the patient has been diagnosed with at least one disease or disorder in the group and is zero if the patient has not be diagnosed with at least one disease or disorder in the group.

In some embodiments, a value of the first variable is zero or one based on the indication of whether the measured level of mean corpuscular hemoglobin fell in outside normal range. For example, the value of the first variable is 0 if the measured level fell in the normal range and is 1 if the measured level fell outside the normal range. A value of the second variable is zero or one based on the indication of whether the measured level of whole blood potassium fell outside the normal range. For example, the value of the first variable is 0 if the measured level fell in the normal range and is 1 if the measured level fell outside the normal range. A value of the third variable is zero or one based on whether the patient has been diagnosed with at least one disease or disorder in the group. For example, the value of the third variable is 1 if the patient has been diagnosed with at least one disease or disorder in the group and is 0 if the patient has not be diagnosed with at least one disease or disorder in the group. In some embodiments, the coefficients of the statistical regression model are as follows: intercept=0.51; first variable=−2.6; second variable=−1.9; and third variable=1.2.

In some embodiments, a value of the first variable is zero or one based on the indication of whether the measured level of mean corpuscular hemoglobin fell outside the normal range. For example, the value of the first variable is 0 if the measured level fell in the normal range and is 1 if the measured level fell outside the normal range. A value of the second variable is zero or one based on the indication of whether the measured level of whole blood potassium fell outside the normal range. For example, the value of the second variable is 0 if the measured level fell in the normal range and is 1 if the measured level fell outside the normal range. A value of the third variable is zero or one based on whether the patient has been diagnosed with at least one disease or disorder in the group. For example, value of the third variable is 1 if the patient has been diagnosed with at least one disease or disorder in the group and is 0 if the patient has not be diagnosed with at least one disease or disorder in the group. In some embodiments, the coefficients of the statistical regression model fall in the following ranges: 0.3<intercept<0.72; −2.90<first variable<−2.34; −2.42<second variable<−1.31; and 0.89<third variable <1.55.

In some embodiments the accessed and/or received information regarding the patient further comprises an indication of whether a measured level of blood pH for the patient fell outside a normal range for the patient. In some embodiments, the determination of the estimated risk of the patient experiencing septic shock within the specified time period is based, at least in part, on the indication of whether the measured level of mean corpuscular hemoglobin fell outside the normal range, the indication of whether the measured level of whole blood potassium fell outside the normal range, and the indication of whether the measured level of blood pH fell outside the normal range.

In some embodiments, determination of the estimated risk of the patient experiencing septic shock within the specified time period is based on the indication of whether the measured level of mean corpuscular hemoglobin fell outside the normal range, the indication of whether the measured level of whole blood potassium fell outside the normal range, and the indication of whether the measured level of blood pH fell outside the normal range.

In some embodiments, the determination of the estimated risk of the patient experiencing septic shock within the specified time period is based, at least in part, on a statistical model of risk including variables based on whether the measured level of mean corpuscular hemoglobin fell outside the normal range, whether the measured level of whole blood potassium fell outside the normal range, and whether the measured level of blood pH fell outside the normal range.

In some embodiments, the accessed and/or received information regarding the patient further comprises an indication of whether a measured level of blood pH for the patient fell outside a normal range for the patient, an indication of whether mean corpuscular hemoglobin was measured, and an indication of whether whole blood potassium was measured. The determination of the estimated risk of the patient experiencing septic shock within the specified time period is based, at least on part, on the indication of whether the measured level of mean corpuscular hemoglobin fell outside the normal range, the indication of whether the measured level of whole blood potassium fell outside the normal range, the indication of whether the measured level of blood pH fell outside the normal range, the indication of whether mean corpuscular hemoglobin was measured, and the indication of whether whole blood potassium was measured.

In some embodiments, the determination of the estimated risk of the patient experiencing septic shock within the specified time period is based on the indication of whether the measured level of mean corpuscular hemoglobin fell outside the normal range, the indication of whether the measured level of whole blood potassium fell outside the normal range, the indication of whether the measured level of blood pH fell outside the normal range, the indication of whether mean corpuscular hemoglobin was measured, and the indication of whether whole blood potassium was measured.

In some embodiments, the determination of the estimated risk of the patient experiencing septic shock within the specified time period is based, at least in part, on a statistical model of risk including variables based on whether the measured level of mean corpuscular hemoglobin fell outside the normal range, whether the measured level of whole blood potassium fell outside the normal range, whether the measured level of blood pH fell outside the normal range, whether mean corpuscular hemoglobin was measured, and whether whole blood potassium was measured.

In some embodiments, the accessed and/or received information regarding the patient further includes an indication of whether a measured level of blood pH for the patient fell outside a normal range for the patient, an indication of whether the measured level of blood pH was measured, an indication of whether mean corpuscular hemoglobin was measured, and an indication of whether whole blood potassium was measured. The determination of the estimated risk of the patient experiencing septic shock within the specified time period is based, at least in part, on the indication of whether the measured level of mean corpuscular hemoglobin fell outside the normal range, the indication of whether the measured level of whole blood potassium fell outside the normal range, the indication of whether the measured level of blood pH fell outside the normal range, the indication of whether the measured level of blood pH was measured, the indication of whether mean corpuscular hemoglobin was measured, and the indication of whether whole blood potassium was measured.

In some embodiments, the determination of the estimated risk of the patient experiencing septic shock within the specified time period is based on the indication of whether the measured level of mean corpuscular hemoglobin fell outside the normal range, the indication of whether the measured level of whole blood potassium fell outside the normal range, the indication of whether the measured level of blood pH fell outside the normal range, the indication of whether the measured level of blood pH was measured, the indication of whether mean corpuscular hemoglobin was measured, and the indication of whether whole blood potassium was measured.

In some embodiments, the determination of the estimated risk of the patient experiencing septic shock within the specified time period is based, at least in part, on a statistical model of risk including variables based on whether the measured level of mean corpuscular hemoglobin fell outside the normal range, whether the measured level of whole blood potassium fell outside the normal range, whether the measured level of blood pH fell outside the normal range, whether mean corpuscular hemoglobin was measured, whether whole blood potassium was measured, and whether the blood pH was measured.

In some embodiments, a value of a first variable is zero or one based on the indication of whether the measured level of mean corpuscular hemoglobin fell outside the normal range. For example, the value of the first variable is 1 where the measured level of mean corpuscular hemoglobin fell outside the normal range and zero where the measured level of mean corpuscular hemoglobin fell within the normal range or was not measured. A value of a second variable is zero or one based on the indication of whether the mean corpuscular hemoglobin was measured. For example, the second variable is 1 where the mean corpuscular hemoglobin was not measured and is 0 where the mean corpuscular hemoglobin was measured. A value of a third variable based on the indication of whether the measured level of blood pH fell outside the normal range. For example, value of the third variable is 1 where the measured level of blood pH fell outside the normal range and is zero where the measured level of blood pH fell within the normal range or was not measured. A value of a fourth variable is zero or one based on whether the blood pH was measured. For example, the value of the fourth variable is 1 where the blood pH was not measured and is 0 where the blood pH was measured. A value of a fifth variable is zero or one based on the indication of whether the measured level of whole blood potassium fell outside the normal range. For example, value of the fifth variable is 1 where the measured level of whole blood potassium fell outside the normal range and is zero where the measured level of whole blood potassium value fell within the normal range or was not measured. A value of a sixth variable is zero or one based on whether the whole blood potassium was measured is 1 where the whole blood potassium was not measured and is 0 where the whole blood potassium was measured. In some embodiments, the coefficients of the statistical regression model fall in the following ranges: 0.38<first variable<1.08; 2.57<second variable<3.31; 1.04<third variable<1.9; −0.57<fourth variable<−0.07; 0.81<fifth variable<2.13; and 0.77<sixth variable<1.75. In some embodiments, the coefficients of the statistical regression model fall in the following ranges: 0.58<first variable<0.91; 2.75<second variable<3.13; 1.25<third variable<1.69; −0.44<fourth variable<−0.20; 1.14<fifth variable<1.8; and 1.01<sixth variable<1.51. In some embodiments, the first variable is about 0.73, the second variable is about 2.94, the third variable is about 1.47, the fourth variable is about −0.32, the fifth variable is about 1.47, and the sixth variable is about 1.26.

In some embodiments, the method may include a determination that the intercept is zero.

In some embodiments, where the estimated risk is above a threshold value, the method may include providing an alert of a high risk of the patient experiencing septic shock within the specified time period. In some embodiments, providing the alert comprises displaying the alert on a display device. In some embodiments, providing the alert comprises transmitting the alert to one or more care providers for the patient.

In someone embodiments, the information regarding the estimated risk of the patient experiencing septic shock within the specified time period is provided to a clinical decision support system as factor in determining a treatment or care recommendation. In some embodiments, where the estimated risk the patient experiencing septic shock is above a threshold value, the method includes providing information to a clinical decision support system that the patient is at increased risk of septic shock.

In some embodiments, the method is a method of identifying a patient at increased risk of septic shock, and wherein the method further comprises determining if the estimated risk is above a threshold value, and identifying the patient as having an increased risk of septic shock where the estimated risk is above the threshold value.

In some embodiments, the method of identifying a patient at increased risk of septic shock includes detecting a level of mean corpuscular hemoglobin in the patient's blood and identifying whether the detected level of mean corpuscular hemoglobin the patient's blood falls outside a normal range for the patient. The method also includes detecting a level of whole blood potassium for the patient and identifying whether the detected level of whole blood potassium for the patient falls outside a normal range for the patient. The method also includes identifying whether the patient has had a diagnosis of at least one disease or disorder on a group of diseases or disorders. The method further includes determining an estimated risk that the patient will experience septic shock within a specified time period based on the identification of whether the detected level of mean corpuscular hemoglobin falls outside the normal range, the identification of whether the detected level of whole blood potassium falls outside the normal range, and whether patient has had a diagnosis of at least one disease or disorder from the group of diseases and disorders, and where the estimated risk is above a threshold value, identifying the patient as having an increased risk of septic shock.

In some embodiments, the determination of the estimated risk of the patient experiencing septic shock within the specified time period is based, at least in part, on a statistical model of risk using a first variable based on the identification of whether the measured level of mean corpuscular hemoglobin fell outside the normal range, a second variable based on the identification of whether the measured level of whole blood potassium fell outside the normal range, and a third variable based on whether the patient has been diagnosed with at least one disease or disorder in the group.

In some embodiments, the determination of the estimated risk of the patient experiencing septic shock within the specified time period is based on a statistical model of risk using a first variable based on the identification of whether the measured level of mean corpuscular hemoglobin fell outside the normal range, a second variable based on the identification of whether the measured level of whole blood potassium fell outside the normal range, and a third variable based on whether the patient has been diagnosed with at least one disease or disorder in the group. In some embodiments, the model is a statistical regression model based on the first variable, the second variable, and the third variable. In some embodiments, a value of the first variable is zero or non-zero based on the identification of whether the measured level of mean corpuscular hemoglobin fell outside the normal range. For example, the value of the first variable is zero if the measured level fell in the normal range and is nonzero if the measured level fell outside the normal range. A value of the second variable is zero or non-zero based on the identification of whether the measured level of whole blood potassium fell outside the normal rage. For example, the value of the second variable is zero if the measured level fell in the normal range and is nonzero if the measured level fell outside the normal range. A value of the third variable is zero or non-zero based on whether the patient has been diagnosed with at least one disease or disorder in the group. For example, value of the third variable is nonzero if the patient has been diagnosed with at least one disease or disorder in the group and is zero if the patient has not be diagnosed with at least one disease or disorder in the group. In some embodiments, the coefficients of the statistical regression model are as follows: intercept=0.51; first variable=−2.6; second variable=−1.9; and third variable=1.2. In some embodiments, the coefficients of the statistical regression model fall in the following ranges: 0.3<intercept<0.72; −2.90<first variable<−2.34; −2.42<second variable<−1.31; and 0.89<third variable<1.55.

In some embodiments, the method of identifying a patient at increased risk of septic shock includes detecting a level of mean corpuscular hemoglobin in the patient's blood and identifying whether the detected level of mean corpuscular hemoglobin the patient's blood falls outside a normal range for the patient, detecting a level of whole blood potassium for the patient and identifying whether the detected level of whole blood potassium for the patient falls outside a normal range for the patient, and detecting a level of blood pH for the patient and identifying whether the detected level of blood pH falls outside a normal range for the patient. The method also includes determining an estimated risk that the patient will experience septic shock within a specified time period based on the identification of whether the detected level of mean corpuscular hemoglobin falls outside the normal range, the identification of whether the detected level of whole blood potassium falls outside the normal range, and the identification of whether the detected level of blood pH falls outside a normal range for the patient, and where the estimated risk is above a threshold value, identifying the patient as having an increased risk of septic shock.

In some embodiments, the determination of the estimated risk of the patient experiencing septic shock within the specified time period is based, at least in part, on a statistical model of risk using a first variable based on the identification of whether the detected level of mean corpuscular hemoglobin fell outside the normal range, a second variable based on the identification of whether the detected level of whole blood potassium fell outside the normal range, and a third variable based on whether the detected level of blood pH fell outside a normal range.

In some embodiments, the method of identifying a patient at increased risk of septic shock, includes accessing information regarding a patient, the information including a measured level of mean corpuscular hemoglobin, a measured level of whole blood potassium, and whether the patient has been diagnosed with at least one disease or disorder from a list of diseases and disorders. The method also includes determining, via one or more microprocessors, an estimated risk of the patient experiencing septic shock within a specified time period based on, at least, the measured level of mean corpuscular hemoglobin, the measured level of whole blood potassium, and whether the patient has been diagnosed with at least one disease or disorder from a list of diseases and disorders, and where the estimated risk of septic shock is higher than a threshold level, identifying the patient as being at increased risk of septic shock.

In some embodiments, the group of diseases or disorders comprises: hypersmolality, hypernatremia, acidosis, alkalosis, mixed-acid based balance disorder, fluid overload, electrolyte and fluid disorders, and angioneurotic edema.

In some embodiments, the group of diseases or disorders consists of: hypersmolality, hypernatremia, acidosis, alkalosis, mixed-acid based balance disorder, fluid overload, electrolyte and fluid disorders, and angioneurotic edema.

In some embodiments, the group of diseases or disorders comprises diseases or disorders falling in the following International Classification of Disease 9 Clinically Modified (ICD-9-CM) Codes: 2760, 2762, 2763, 2764, 2766, 27669, 2769, and 9951.

In some embodiments, the group of diseases or disorders consists of diseases or disorders falling in the following International Classification of Disease 9 Clinically Modified (ICD-9-CM) Codes: 2760, 2762, 2763, 2764, 2766, 27669, 2769, and 9951.

In some embodiments, the group of diseases or disorders comprises disorders related to electrolyte balance and fluid balance.

In some embodiments, the specified time period is less than 36 hours. In some embodiments, the specified time period includes 24 hours. In some embodiments, the specified time period falls in a range of 3 hours and 25 hours.

In some embodiments, the patient is in an intensive care unit and wherein the determining of the estimated risk is specific to patients in an intensive care unit.

Some embodiments provide a non-transitory computer readable medium including executable instructions, that, when executed by one or more processors, perform the methods described herein.

Some embodiments provide a system including a database configured to store information regarding a patient including an indication of whether a measured level of mean corpuscular hemoglobin for the patient fell outside a normal range for the patient, an indication of whether a measured level of whole blood potassium for the patient fell outside a normal range for the patient, and information regarding current and prior diagnoses of diseases and disorders for the patient. In some embodiments, the system also includes a septic shock risk assessment module configured to receive or access the information regarding the patient from the database, determine whether the patient has been diagnosed with one or more diseases and disorders in a group of diseases and disorders, and determine an estimated risk of the patient experiencing septic shock within a specified time period based on, at least, the indication of whether the measured level of mean corpuscular hemoglobin fell outside the normal range, the indication of whether the measured level of whole blood potassium fell outside the normal range, and the determination of whether the patient has been diagnosed with the at least one disease or disorder in the group.

Some embodiments provide a system including a database configured to store information regarding a patient including an indication of whether a measured level of mean corpuscular hemoglobin for the patient fell outside a normal range for the patient, an indication of whether a measured level of whole blood potassium for the patient fell outside a normal range for the patient, and an indication of whether a measured level of blood pH for the patient fell outside a normal range. In some embodiments, the system also includes a septic shock risk assessment module configured to receive or access the information regarding the patient from the database, determine whether the patient has been diagnosed with one or more diseases and disorders in a group of diseases and disorders, and determine an estimated risk of the patient experiencing septic shock within a specified time period based on, at least, the indication of whether the measured level of mean corpuscular hemoglobin fell outside the normal range, the indication of whether the measured level of whole blood potassium fell outside the normal range, and the determination of whether the measured level of blood pH feel outside the normal range.

Some embodiments provide a system including a database configured to store information regarding a patient including an indication of whether a measured level of mean corpuscular hemoglobin for the patient fell outside a normal range for the patient or an indication that mean corpuscular hemoglobin has not been measured for the patient, an indication of whether a measured level of whole blood potassium for the patient fell outside a normal range for the patient or an indication that whole blood potassium has not been measured for the patient, and an indication of whether a measured level of blood pH for the patient fell outside a normal range or an indication that blood pH has not been measured for the patient. In some embodiments, the system also includes a septic shock risk assessment module configured to receive or access the information regarding the patient from the database, determine an estimated risk of the patient experiencing septic shock within a specified time period based on, at least, the indication of whether the measured level of mean corpuscular hemoglobin fell outside the normal range or the indication that the mean corpuscular hemoglobin was not measured, the indication of whether the measured level of whole blood potassium fell outside the normal range or the indication that the whole blood potassium has not been measured, the indication of whether the measured level of blood pH feel outside the normal range or the indication that the level of blood pH has not been measured.

In some embodiments, the risk assessment module is further configured to determine whether the estimated risk is larger than a threshold value. In some embodiments, the system includes an alert module configured to provide or transmit an alert when the estimated risk for the patient is larger than the threshold value. In some embodiments, the system is a clinical decision support system and information regarding the estimated risk of septic shock is used to determining a treatment or a care recommendation for the patient.

### BRIEF DESCRIPTION OF FIGURES

Various embodiments of the present disclosure will be described herein below with reference to the figures wherein:

FIG. 1 schematically depicts a process for developing a predictive model for early identification of increased risk of septic shock and application of the model to improve patient care or outcomes in accordance with some embodiments;

FIG. 2 depicts characteristics of some methods of forming models to attempt to identify a person or patient with increased risk of septic shock in accordance with some embodiments;

FIG. 3 schematically depict time lines and the clinical criteria used to identify progression to septic shock in a patient during the patient's stay in the ICU as identified based on electronic medical record data in accordance with some embodiments;

FIG. 4. graphically depicts a generated causal relationship network map based on data 24 hours prior to sepsis diagnosis and an enlarged view of the portion closely connected with sepsis for Example 1 in accordance with some embodiments;

FIG. 5 describes and depicts a logistical regression model, ROC curve and AUC for determination of septic shock based on Example 1 in accordance with some embodiments;

FIG. 6A graphically depicts a generated causal relationship network map based on data 24 hours prior to sepsis diagnosis and an enlarged view of a portion of the network including age superimposed on the full network for Example 2 in accordance with some embodiments;

FIG. 6B graphically depicts a subnetwork of the network of FIG. 6A including nodes closely connected to septic shock in accordance with some embodiments;

FIG. 7 describes and depicts a logistical regression model, ROC curve and AUC for determination of septic shock based on Example 2 in accordance with some embodiments;

FIG. 8A is a network diagram schematically depicting an example system that may be used in part or in full in a septic shock risk assessment system or method in accordance with some embodiments;

FIG. 8B is a block diagram including modules that may be employed to implement some aspects of some embodiments described herein; and

FIG. 9 is a block diagram of an exemplary computer system that may be used in a septic shock risk assessment system in accordance with some embodiments.

### DETAILED DESCRIPTION

Embodiments described herein include methods for early prediction of sepsis and increased risk of septic shock, methods and system for providing an alert or warning regarding an increased risk of developing septic shock, and a clinical decision support system for patient care that provides an alert or warning regarding an increased risk of developing septic shock.

In some embodiments, method and systems described herein can be embedded into clinical software at a point of care (e.g., an intensive care unit (ICU)). In such embodiments, care providers could then use patient specific risk estimates for septic shock that are provided through clinical software to inform patient care in the ICU. In some embodiments, the method or system provides an alert to care providers regarding a patient at increased risk of septic shock.

Early identification of septic shock (or risk of septic shock) is extremely valuable in clinical settings given the high mortality rates associated with this condition. Currently, generally speaking, no molecular diagnostics for sepsis are available and diagnosis is entirely based on clinical observation and evaluation by the clinician, increasing the difficulty of providing accurate estimates for a risk of septic shock in a clinical setting using conventional methods.

Further, there is a strong unmet need for a reliable clinical tool or system that can be used for large scale automated screening to identify high-risk patients for sepsis or septic shock.

Some embodiments of systems and methods that identify patients at high risk of sepsis or septic shock enable both high rates of early diagnosis of septic shock and more efficient utilization of often scarce clinical resources. Some embodiments enable targeted close monitoring of a smaller subset of patients (e.g., of patients in an ICU), making it both practicable and invaluable in the ICU setting.

Methods

In accordance with some embodiments, a method includes accessing and/or receiving information regarding a patient. In some embodiments, at least some of the accessed and/or received information is stored in a database. In some embodiments, all of the accessed and/or received information is stored in one or more databases. In some embodiments, the accessed and/or received information includes an indication of whether a measured level of mean corpuscular hemoglobin for the patient fell outside a normal range for the patient and an indication of whether a measured level of whole blood potassium for the patient fell outside a normal range for the patient. One or both of the normal range for a measured level of mean corpuscular hemoglobin and the normal range for a measured level of whole blood potassium may not be the same for every patient, and may vary based on patient-specific factors, such as age. Accordingly, the normal range for the patient is a reference range corresponding to any relevant patient-specific factors, such as age. Further, the normal range for each measurement may be specific to the type of equipment used to obtain the measurement. In some embodiments, the normal range for the patient can be stored in a database. The method also includes determining, via one or more microprocessors, an estimated risk of the patient experiencing septic shock within a specified time period based on, at least, the indication of whether the measured level of mean corpuscular hemoglobin fell outside the normal range and the indication of whether the measured level of whole blood potassium fell outside the normal range. The method also includes providing information regarding the estimated risk of the patient experiencing septic shock within the specified time period. In some embodiments, the information regarding the estimated risk of the patient experiencing septic shock within the specified time period is saved to a database and/or saved in the patient's electronic medical record.

In some embodiments, the accessed and/or received information regarding the patient further includes information regarding the whether the patient has been diagnosed with at least one disease or disorder in a group of diseases and disorders, and determining the estimated risk of the patient experiencing septic shock within the specified time period is based on, at least, the indication of whether the measured level of mean corpuscular hemoglobin fell outside the normal range, the indication of whether the measured level of whole blood potassium fell outside the normal range, and the information regarding whether the patient has been diagnosed with at least one disease or disorder in the group.

In some embodiments, the determining of the estimated risk of the patient experiencing septic shock within the specified time period is based on the indication of whether the measured level of mean corpuscular hemoglobin fell outside the normal range, the indication of whether the measured level of whole blood potassium fell outside the normal range, and the information regarding whether the patient has been diagnosed with at least one disease or disorder in the group.

In some embodiments, the determining of the estimated risk of the patient experiencing septic shock within the specified time period is based, at least in part, on a statistical model of risk using a first variable based on the indication of whether the measured level of mean corpuscular hemoglobin fell outside the normal range, a second variable based on the indication of whether the measured level of whole blood potassium fell outside the normal range, and a third variable based on whether the patient has been diagnosed with at least one disease or disorder in the group.

In some embodiments, the determining of the estimated risk of the patient experiencing septic shock within the specified time period is based on a statistical model of risk using a first variable based on the indication of whether the measured level of mean corpuscular hemoglobin fell outside the normal range, a second variable based on the indication of whether the measured level of whole blood potassium fell outside the normal range, and a third variable based on whether the patient has been diagnosed with at least one disease or disorder in the group.

In some embodiments, the group of diseases or disorders includes: hypersmolality, hypernatremia, acidosis, alkalosis, mixed-acid based balance disorder, fluid overload, electrolyte and fluid disorders, and angioneurotic edema.

In some embodiments, the group of diseases or disorders consists of: hypersmolality, hypernatremia, acidosis, alkalosis, mixed-acid based balance disorder, fluid overload, electrolyte and fluid disorders, and angioneurotic edema.

In some embodiments, the group of diseases or disorders includes diseases or disorders falling in the following International Classification of Disease 9 Clinically Modified (ICD-9-CM) Codes: 2760, 2762, 2763, 2764, 2766, 27669, 2769, and 9951.

In some embodiments, the group of diseases or disorders consists of diseases or disorders falling in the following International Classification of Disease 9 Clinically Modified (ICD-9-CM) Codes: 2760, 2762, 2763, 2764, 2766, 27669, 2769, and 9951.

In some embodiments, the group of diseases or disorders comprises disorders related to electrolyte balance and fluid balance.

In some embodiments, the model is a statistical regression model based on the first variable, the second variable, and the third variable.

In some embodiments, a value of the first variable based on the indication of whether the measured level of mean corpuscular hemoglobin fell outside the normal range is zero if the measured level fell in the normal range and is nonzero if the measured level fell outside the normal range, a value of the second variable based on the indication of whether the measured level of whole blood potassium fell outside the normal range is zero if the measured level fell in the normal range and is nonzero if the measured level fell outside the normal range, and a value of the third variable based on whether the patient has been diagnosed with at least one disease or disorder in the group is nonzero if the patient has been diagnosed with at least one disease or disorder in the group and is zero if the patient has not be diagnosed with at least one disease or disorder in the group.

In some embodiments, a value of the first variable based on the indication of whether the measured level of mean corpuscular hemoglobin fell outside the normal range is 0 if the measured level fell in the normal range and is 1 if the measured level fell outside the normal range, a value of the second variable based on the indication of whether the measured level of whole blood potassium fell outside the normal range is 0 if the measured level fell in the normal range and is 1 if the measured level fell outside the normal range, a value of the third variable based on whether the patient has been diagnosed with at least one disease or disorder in the group is 1 if the patient has been diagnosed with at least one disease or disorder in the group and is 0 if the patient has not be diagnosed with at least one disease or disorder in the group; and the coefficients of the statistical regression model are as follows: intercept=0.51; first variable=−2.6; second variable=−1.9; and third variable=1.2.

In some embodiments, a value of the first variable based on the indication of whether the measured level of mean corpuscular hemoglobin fell outside the normal range is 0 if the measured level fell in the normal range and is 1 if the measured level fell outside the normal range, a value of the second variable based on the indication of whether the measured level of whole blood potassium fell outside the normal range is 0 if the measured level fell in the normal range and is 1 if the measured level fell outside the normal range, a value of the third variable based on whether the patient has been diagnosed with at least one disease or disorder in the group is 1 if the patient has been diagnosed with at least one disease or disorder in the group and is 0 if the patient has not be diagnosed with at least one disease or disorder in the group; and the coefficients of the statistical regression model fall in the following ranges: 0.3<intercept<0.72; −2.90<first variable<−2.34; −2.42<second variable<−1.31; and 0.89<third variable<1.55.

In some embodiments, the accessed and/or received information regarding the patient further comprises an indication of whether a measured level of blood pH for the patient fell outside a normal range for the patient. In some embodiments, the determining of the estimated risk of the patient experiencing septic shock within the specified time period is based, at least in part, on the indication of whether the measured level of mean corpuscular hemoglobin fell outside the normal range, the indication of whether the measured level of whole blood potassium fell outside the normal range, and the indication of whether the measured level of blood pH fell outside the normal range. In some embodiments, the determining of the estimated risk of the patient experiencing septic shock within the specified time period is based on the indication of whether the measured level of mean corpuscular hemoglobin fell outside the normal range, the indication of whether the measured level of whole blood potassium fell outside the normal range, and the indication of whether the measured level of blood pH fell outside the normal range.

In some embodiments, the determining of the estimated risk of the patient experiencing septic shock within the specified time period is based, at least in part, on a statistical model of risk including variables based on whether the measured level of mean corpuscular hemoglobin fell outside the normal range, whether the measured level of whole blood potassium fell outside the normal range, and whether the measured level of blood pH fell outside the normal range.

In some embodiments, the accessed and/or received information regarding the patient further comprises an indication of whether a measured level of blood pH for the patient fell outside a normal range for the patient, an indication of whether mean corpuscular hemoglobin was measured, and an indication of whether whole blood potassium was measured. In some embodiments, the determining of the estimated risk of the patient experiencing septic shock within the specified time period is based, at least on part, on the indication of whether the measured level of mean corpuscular hemoglobin fell outside the normal range, the indication of whether the measured level of whole blood potassium fell outside the normal range, the indication of whether the measured level of blood pH fell outside the normal range, the indication of whether mean corpuscular hemoglobin was measured, and the indication of whether whole blood potassium was measured. In some embodiments, the determining of the estimated risk of the patient experiencing septic shock within the specified time period is based on the indication of whether the measured level of mean corpuscular hemoglobin fell outside the normal range, the indication of whether the measured level of whole blood potassium fell outside the normal range, the indication of whether the measured level of blood pH fell outside the normal range, the indication of whether mean corpuscular hemoglobin was measured, and the indication of whether whole blood potassium was measured.

In some embodiments, the determining of the estimated risk of the patient experiencing septic shock within the specified time period is based, at least in part, on a statistical model of risk including variables based on whether the measured level of mean corpuscular hemoglobin fell outside the normal range, whether the measured level of whole blood potassium fell outside the normal range, whether the measured level of blood pH fell outside the normal range, whether mean corpuscular hemoglobin was measured, and whether whole blood potassium was measured.

In some embodiments, the accessed and/or received information regarding the patient further comprises an indication of whether a measured level of blood pH for the patient fell outside a normal range for the patient, an indication of whether the measured level of blood pH was measured, an indication of whether mean corpuscular hemoglobin was measured, and an indication of whether whole blood potassium was measured. In some embodiments, the determining of the estimated risk of the patient experiencing septic shock within the specified time period is based, at least in part, on the indication of whether the measured level of mean corpuscular hemoglobin fell outside the normal range, the indication of whether the measured level of whole blood potassium fell outside the normal range, the indication of whether the measured level of blood pH fell outside the normal range, the indication of whether the measured level of blood pH was measured, the indication of whether mean corpuscular hemoglobin was measured, and the indication of whether whole blood potassium was measured.

In some embodiments, the determining of the estimated risk of the patient experiencing septic shock within the specified time period is based on the indication of whether the measured level of mean corpuscular hemoglobin fell outside the normal range, the indication of whether the measured level of whole blood potassium fell outside the normal range, the indication of whether the measured level of blood pH fell outside the normal range, the indication of whether the measured level of blood pH was measured, the indication of whether mean corpuscular hemoglobin was measured, and the indication of whether whole blood potassium was measured.

In some embodiments, the determining of the estimated risk of the patient experiencing septic shock within the specified time period is based, at least in part, on a statistical model of risk including variables based on whether the measured level of mean corpuscular hemoglobin fell outside the normal range, whether the measured level of whole blood potassium fell outside the normal range, whether the measured level of blood pH fell outside the normal range, whether mean corpuscular hemoglobin was measured, whether whole blood potassium was measured, and whether the blood pH was measured. In some embodiments, a value of a first variable based on the indication of whether the measured level of mean corpuscular hemoglobin fell outside the normal range is 1 where the measured level of mean corpuscular hemoglobin fell outside the normal range and zero where the measured level of mean corpuscular hemoglobin fell within the normal range or was not measured, a value of a second variable based on the indication of whether the mean corpuscular hemoglobin was measured is 1 where the mean corpuscular hemoglobin was not measured and is 0 where the mean corpuscular hemoglobin was measured, a value of a third variable based on the indication of whether the measured level of blood pH fell outside the normal range is 1 where the measured level of blood pH fell outside the normal range and is zero where the measured level of blood pH fell within the normal range or was not measured, a value of a fourth variable based on whether the blood pH was measured is 1 where the blood pH was not measured and is 0 where the blood pH was measured; a value of a fifth variable based on the indication of whether the measured level of whole blood potassium fell outside the normal range is 1 where the measured level of whole blood potassium fell outside the normal range and is zero where the measured level of whole blood potassium value fell within the normal range or was not measured, a value of a sixth variable based on whether the whole blood potassium was measured is 1 where the whole blood potassium was not measured and is 0 where the whole blood potassium was measured, and wherein the coefficients of the statistical regression model fall in the following ranges: 0.38<first variable<1.08; 2.57<second variable<3.31; 1.04<third variable<1.9; −0.57<fourth variable<−0.07; 0.81<fifth variable<2.13; and 0.77<sixth variable<1.75. In some embodiments, the coefficients of the statistical regression model fall in the following ranges: 0.58<first variable<0.91; 2.75<second variable<3.13; 1.25<third variable<1.69; −0.44<fourth variable<−0.20; 1.14<fifth variable<1.8; and 1.01<sixth variable<1.51. In some embodiments, the first variable is about 0.73, the second variable is about 2.94, the third variable is about 1.47, the fourth variable is about −0.32, the fifth variable is about 1.47, and the sixth variable is about 1.26. In some embodiments, the intercept is zero.

In some embodiments, the method includes where the estimated risk is above a threshold value, providing an alert of a high risk of the patient experiencing septic shock within the specified time period. In some embodiments, providing the alert includes displaying the alert on a display device. In some embodiments, providing the alert comprises transmitting the alert to one or more care providers for the patient.

In some embodiments, information regarding the estimated risk of the patient experiencing septic shock within the specified time period is provided to a clinical decision support system as factor in determining a treatment or care recommendation.

In some embodiments, the method also includes where the estimated risk the patient experiencing septic shock is above a threshold value, providing information to a clinical decision support system that the patient is at increased risk of septic shock.

In some embodiments, the method is a method of identifying a patient at increased risk of septic shock, and the method further comprises determining if the estimated risk is above a threshold value, and identifying the patient as having an increased risk of septic shock where the estimated risk is above the threshold value.

Some embodiments provide a method of identifying a patient at increased risk of septic shock. The method includes detecting a level of mean corpuscular hemoglobin in the patient's blood and identifying whether the detected level of mean corpuscular hemoglobin the patient's blood falls outside a normal range for the patient; detecting a level of whole blood potassium for the patient and identifying whether the detected level of whole blood potassium for the patient falls outside a normal range for the patient; and detecting a level of blood pH for the patient and identifying whether the detected level of blood pH falls outside a normal range for the patient. the method also includes determining an estimated risk that the patient will experience septic shock within a specified time period based on the identification of whether the detected level of mean corpuscular hemoglobin falls outside the normal range, the identification of whether the detected level of whole blood potassium falls outside the normal range, and the identification of whether the detected level of blood pH falls outside a normal range for the patient; and where the estimated risk is above a threshold value, identifying the patient as having an increased risk of septic shock. In some embodiments, the determining of the estimated risk of the patient experiencing septic shock within the specified time period is based, at least in part, on a statistical model of risk using a first variable based on the identification of whether the detected level of mean corpuscular hemoglobin fell outside the normal range, a second variable based on the identification of whether the detected level of whole blood potassium fell outside the normal range, and a third variable based on whether the detected level of blood pH fell outside a normal range.

Some embodiments provide a method of identifying a patient at increased risk of septic shock. The method includes detecting a level of mean corpuscular hemoglobin in the patient's blood and identifying whether the detected level of mean corpuscular hemoglobin the patient's blood falls within a normal range for the patient. The method also includes detecting a level of whole blood potassium for the patient and identifying whether the detected level of whole blood potassium for the patient falls within a normal range for the patient. The method includes identifying whether the patient has had a diagnosis of at least one disease or disorder on a group of diseases or disorders. The method includes determining an estimated risk that the patient will experience septic shock within a specified time period based on the identification of whether the detected level of mean corpuscular hemoglobin falls within the normal range, the identification of whether the detected level of whole blood potassium falls within the normal range, and whether patient has had a diagnosis of at least one disease or disorder from the group of diseases and disorders. The method also includes where the estimated risk is above a threshold value, identifying the patient as having an increased risk of septic shock.

In some embodiments, determining the estimated risk of the patient experiencing septic shock within the specified time period is based, at least in part, on a statistical model of risk using a first variable based on the identification of whether the measured level of mean corpuscular hemoglobin fell outside the normal range, a second variable based on the identification of whether the measured level of whole blood potassium fell outside the normal range, and a third variable based on whether the patient has been diagnosed with at least one disease or disorder in the group.

In some embodiments, determining the estimated risk of the patient experiencing septic shock within the specified time period is based on a statistical model of risk using a first variable based on the identification of whether the measured level of mean corpuscular hemoglobin fell outside the normal range, a second variable based on the identification of whether the measured level of whole blood potassium fell outside the normal range, and a third variable based on whether the patient has been diagnosed with at least one disease or disorder in the group.

In some embodiments, the model is a statistical regression model based on the first variable, the second variable, and the third variable.

In some embodiments a value of the first variable based on the identification of whether the measured level of mean corpuscular hemoglobin fell outside the normal range is zero if the measured level fell in the normal range and is nonzero if the measured level fell outside the normal range, a value of the second variable based on the identification of whether the measured level of whole blood potassium fell in the normal rage is zero if the measured level fell outside the normal range and is nonzero if the measured level fell outside the normal range, and a value of the third variable based on whether the patient has been diagnosed with at least one disease or disorder in the group is nonzero if the patient has been diagnosed with at least one disease or disorder in the group and is zero if the patient has not be diagnosed with at least one disease or disorder in the group.

In some embodiments, a value of the first variable based on the identification of whether the measured level of mean corpuscular hemoglobin fell outside the normal range is 0 if the measured level fell in the normal range and is 1 if the measured level fell outside the normal range, the second variable based on the identification of whether the measured level of whole blood potassium fell outside the normal range is 0 if the measured level fell in the normal range and is 1 if the measured level fell outside the normal range, the third variable based on whether the patient has been diagnosed with at least one disease or disorder in the group is 1 if the patient has been diagnosed with at least one disease or disorder in the group and is 0 if the patient has not be diagnosed with at least one disease or disorder in the group, and the coefficients of the statistical regression model are as follows: intercept=0.51; first variable=−2.6; second variable=−1.9; and third variable=1.2.

In some embodiments, a value of the first variable based on the identification of whether the measured level of mean corpuscular hemoglobin fell outside the normal range is 0 if the measured level fell in the normal range and is 1 if the measured level fell outside the normal range, a value of the second variable based on the identification of whether the measured level of whole blood potassium fell outside the normal range is 0 if the measured level fell in the normal range and is 1 if the measured level fell outside the normal range, a value of the third variable based on whether the patient has been diagnosed with at least one disease or disorder in the group is 1 if the patient has been diagnosed with at least one disease or disorder in the group and is 0 if the patient has not be diagnosed with at least one disease or disorder in the group, and the coefficients of the statistical regression model fall in the following ranges: 0.3<intercept<0.72; −2.90<first variable<−2.34; −2.42<second variable<−1.31; and 0.89<third variable<1.55.

Some embodiments provide a method of identifying a patient at increased risk of septic shock. The method includes accessing information regarding a patient, the information including a measured level of mean corpuscular hemoglobin, a measured level of whole blood potassium, and whether the patient has been diagnosed with at least one disease or disorder from a list of diseases and disorders, and determining, via one or more microprocessors, an estimated risk of the patient experiencing septic shock within a specified time period based on, at least, the measured level of mean corpuscular hemoglobin, the measured level of whole blood potassium, and whether the patient has been diagnosed with at least one disease or disorder from a list of diseases and disorders. The method also includes where the estimated risk of septic shock is higher than a threshold level, identifying the patient as being at increased risk of septic shock.

The specified time period is less than 36 hours in accordance with some embodiments. The specified time period includes 24 hours in accordance with some embodiments. The specified time period falls in a range of 3 hours and 25 hours in accordance with some embodiments.

In accordance with some embodiments, the patient is in an intensive care unit and the determining of the estimated risk is specific to patients in an intensive care unit.

Embodiments also include a non-transitory computer readable medium storing instructions, that, when executed by one or more processors, perform any of the methods described or claimed herein

Systems

Some embodiments also include systems for performing any of the methods described herein.

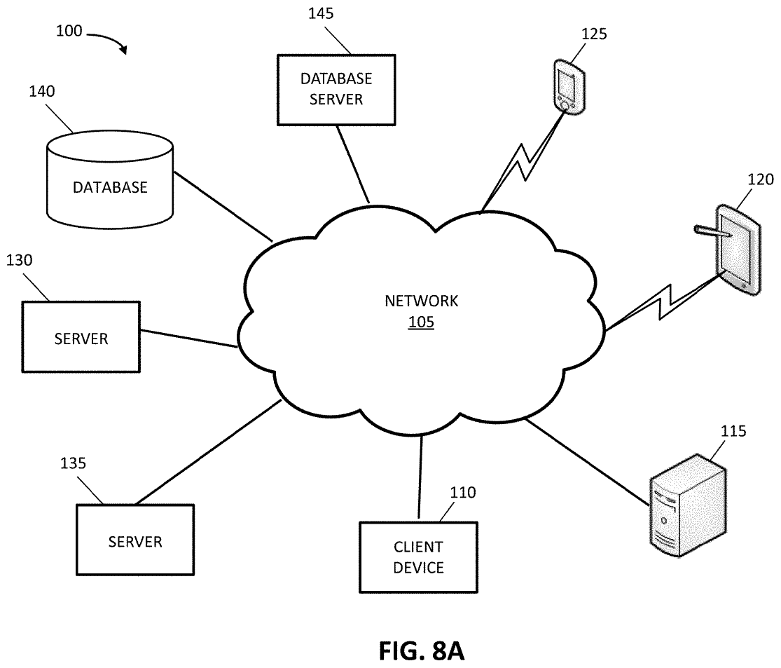

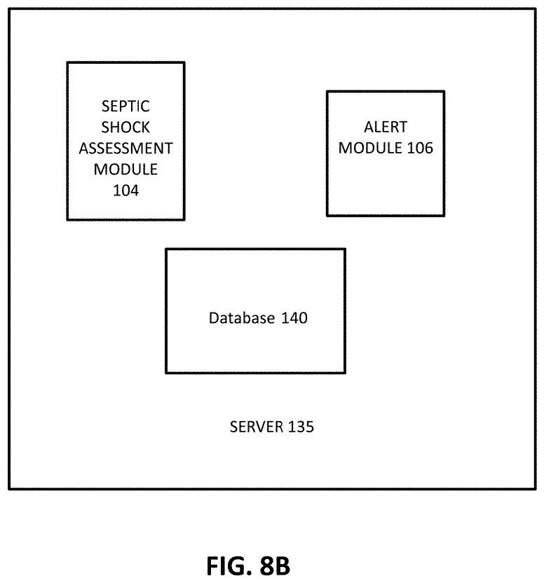

FIG. 8A depicts a network diagram depicting an example system 100 that may be included in part or in full in a septic shock risk assessment system in accordance with some embodiments. Some embodiments provide a system 100 including a database 140 configured to store information regarding a patient including one or more of an indication of whether a measured level of mean corpuscular hemoglobin for the patient fell outside a normal range for the patient, an indication of whether a measured level of whole blood potassium for the patient fell outside a normal range for the patient, information regarding current and prior diagnoses of diseases and disorders for the patient, and an indication regarding whether a measured level of blood pH fell outside a normal range for the patient. In some embodiments, the database 140 is configure to store information regarding a patient including an indication of whether a measured level of mean corpuscular hemoglobin for the patient fell outside a normal range for the patient, an indication of whether a measured level of whole blood potassium for the patient fell outside a normal range for the patient, and one or both of information regarding current and prior diagnoses of diseases and disorders for the patient, and an indication regarding whether a measured level of blood pH fell outside a normal range for the patient. Other elements or aspects of the network diagram are described in more detail below. In some embodiments, the system includes a septic shock assessment module. In some embodiments, the septic shock assessment module may be provided by, executed using or implemented via one or more servers e.g. server 135. FIG. 8B depicts a block diagram of system 100 that includes a septic shock assessment module 104 and an alert module 106. In some embodiments, the server 135 may be in communication, directly or indirectly, with database 140.

In some embodiments, the septic shock risk assessment module 104 is configured to receive or access information regarding the patient from one or more database(s) 140. In some embodiments, the server 135 may be in communication, directly or indirectly, with the one or more database(s) 140.

In some embodiments, the information accessed or received by the septic shock risk assessment module 104 includes one or more of an indication of whether a measured level of mean corpuscular hemoglobin for the patient fell outside a normal range for the patient, an indication of whether a measured level of whole blood potassium for the patient fell outside a normal range for the patient, information regarding current and prior diagnoses of diseases and disorders for the patient, and an indication regarding whether a measured level of blood pH fell outside a normal range for the patient. In some embodiments, the information accessed or received by the septic shock risk assessment module 104 includes an indication of whether a measured level of mean corpuscular hemoglobin for the patient fell outside a normal range for the patient, an indication of whether a measured level of whole blood potassium for the patient fell outside a normal range for the patient, and one or both of information regarding current and prior diagnoses of diseases and disorders for the patient, and an indication regarding whether a measured level of blood pH fell outside a normal range for the patient.

In some embodiments, the septic shock risk assessment module 104 is also configured to determine whether the patient has been diagnosed with one or more diseases and disorders in a group of diseases and disorders. The septic shock risk assessment module 104 is further configured to determine an estimated risk of the patient experiencing septic shock within a specified time period. In some embodiments, this determination is based on, at least, the indication of whether the measured level of mean corpuscular hemoglobin fell outside the normal range, the indication of whether the measured level of whole blood potassium fell outside the normal range, and the determination of whether the patient has been diagnosed with the at least one disease or disorder in the group. In some embodiments, this determination is based on, at least, the indication of whether the measured level of mean corpuscular hemoglobin fell outside the normal range, the indication of whether the measured level of whole blood potassium fell outside the normal range, and the indication of whether the measured level of blood pH fell outside a normal range for the patient. The determination may be performed as described above with respect to the method. The risk assessment module may be further configured to determine whether the estimated risk is larger than a threshold value.

Some embodiments provide a system including a database 140 configured to store information regarding a patient including one or more of an indication of whether a measured level of mean corpuscular hemoglobin for the patient fell outside a normal range for the patient, an indication of whether a measured level of whole blood potassium for the patient fell outside a normal range for the patient, and an indication of whether a measured level of blood pH for the patient fell outside a normal range. The system also includes a septic shock risk assessment module. In some embodiments, the septic shock risk assessment module 104 is configured to receive or access the information regarding the patient from the database 140, determine whether the patient has been diagnosed with one or more diseases and disorders in a group of diseases and disorders, and determine an estimated risk of the patient experiencing septic shock within a specified time period based on, at least, the indication of whether the measured level of mean corpuscular hemoglobin fell outside the normal range, the indication of whether the measured level of whole blood potassium fell outside the normal range, and the determination of whether the measured level of blood pH feel outside the normal range.

Some embodiments provide a system 100 including a database 140 configured to store information regarding a patient including an indication of whether a measured level of mean corpuscular hemoglobin for the patient fell outside a normal range for the patient or an indication that mean corpuscular hemoglobin has not been measured for the patient, an indication of whether a measured level of whole blood potassium for the patient fell outside a normal range for the patient or an indication that whole blood potassium has not been measured for the patient, and an indication of whether a measured level of blood pH for the patient fell outside a normal range or an indication that blood pH has not been measured for the patient. The system also includes a septic shock risk assessment module. In some embodiments, the septic shock risk assessment module 104 is configured to receive or access the information regarding the patient from the database; determine an estimated risk of the patient experiencing septic shock within a specified time period based on, at least, the indication of whether the measured level of mean corpuscular hemoglobin fell outside the normal range or the indication that the mean corpuscular hemoglobin was not measured, the indication of whether the measured level of whole blood potassium fell outside the normal range or the indication that the whole blood potassium has not been measured, the indication of whether the measured level of blood pH feel outside the normal range or the indication that the level of blood pH has not been measured.

In some embodiments, the system further comprises the alert module 106 configured to provide or transmit an alert when the estimated risk for the patient is larger than the threshold value.

In some embodiments, the system is a clinical decision support system and information regarding the estimated risk of septic shock is used to determining a treatment or a care recommendation for the patient. In an example, the clinical decision support system can be implemented in server 130. In some embodiments, or both of the septic shock assessment module 104 and the alert module 106 are in communication with or implemented within a clinical decision support system.

In some embodiments, the system communicates with a clinical decision support system.

In some embodiments, the system or a part of the system may be referred to as a septic shock risk assessment system.

Turning again to FIG. 8A some or all aspects of a septic shock risk assessment system may be implemented in an example system 100. The system 100 can include a network 105, a client device 110, a client device 115, a client device 120, a client device 125, a server 130, a server 135, a database(s) 140, and a database server(s) 145. Each of the client devices 110, 115, 120, 125, server 130, server 135, database(s) 140, and database server(s) 145 is in communication with the network 105.

In an embodiment, one or more portions of network 105 may be an ad hoc network, an intranet, an extranet, a virtual private network (VPN), a local area network (LAN), a wireless LAN (WLAN), a wide area network (W AN), a wireless wide area network (WW AN), a metropolitan area network (MAN), a portion of the Internet, a portion of the Public Switched Telephone Network (PSTN), a cellular telephone network, a wireless network, a WiFi network, a WiMax network, any other type of network, or a combination of two or more such networks.

Examples of a client device include, but are not limited to, work stations, personal computers, general purpose computers, Internet appliances, laptops, desktops, multi-processor systems, set-top boxes, network pes, wireless devices, portable devices, wearable computers, cellular or mobile phones, portable digital assistants (PDAs), smartphones, tablets, ultrabooks, netbooks, multi-processor systems, microprocessor-based or programmable consumer electronics, mini-computers, and the like. Each of client devices 110, 115, 120, 125 may connect to network 105 via a wired or wireless connection.

In an example embodiment, some aspects of the septic shock risk assessment system are included on the client device 110, 115, 120, 125 which may be configured to locally perform some of the functionalities described herein, while the server 130, 135 performs the other functionalities described herein. For example, the client device 110, 115, 120, 125 may receive information regarding the patient and/or receive or display a patient alert, while the server 135 determine an estimated risk of septic shock.

In an alternative embodiment, the client device 110, 115, 120, 125 can perform all the functionalities described herein.

In another alternative embodiment, the septic shock risk assessment system may be primarily implemented on the server 135 and only accessed via the client device 110, 115, 120, 125.

In some embodiments, server 130 and server 135 may be part of a distributed computing environment, where some of the tasks/functionalities are distributed between servers 130 and 135. In some embodiments, server 130 and server 135 are part of a parallel computing environment, where server 130 and server 135 perform tasks/functionalities in parallel.

In some embodiments, each of the server 130, 135, database(s) 140, and database server(s) 145 is connected to the network 105 via a wired connection. Alternatively, one or more of the server 130, 135, database(s) 140, or database server(s) 145 may be connected to the network 105 via a wireless connection. Although not shown, database server(s) 145 can be directly connected to database(s) 140, or servers 130, 135 can be directly connected to the database server(s) 145 and/or database(s) 140. Server 130, 135 comprises one or more computers or processors configured to communicate with client devices 110, 115, 120, 125 via network 105. Server 130, 135 hosts one or more applications or websites accessed by client devices 110, 115, 120, and 125 and/or facilitates access to the content of database(s) 140. Database server(s) 145 comprises one or more computers or processors configured to facilitate access to the content of database(s) 140. Database(s) 140 comprise one or more storage devices for storing data and/or instructions for use by server 130, 135, database server(s) 145, and/or client devices 110, 115, 120, 125. Database(s) 140, servers 130, 135, and/or database server(s) 145 may be located at one or more geographically distributed locations from each other or from client devices 110, 115, 120, 125. Alternatively, database(s) 140 may be included within server 130 or 135, or database server(s) 145.

Certain embodiments are described herein as including logic or a number of components, modules, or mechanisms. Modules may constitute either software modules (e.g., code embodied on a machine-readable medium or in a transmission signal) or hardware modules. A hardware module is a tangible unit capable of performing certain operations and may be configured or arranged in a certain manner. In example embodiments, one or more computer systems (e.g., a standalone, client or server computer system) or one or more hardware modules of a computer system (e.g., a processor or a group of processors) may be configured by software (e.g., an application or application portion) as a hardware module that operates to perform certain operations as described herein.

In various embodiments, a hardware module may be implemented mechanically or electronically. For example, a hardware module may comprise dedicated circuitry or logic that is permanently configured (e.g., as a special-purpose processor, such as a field programmable gate array (FPGA), an application-specific integrated circuit (ASIC), or a Graphics Processing Unit (GPU)) to perform certain operations. A hardware module may also comprise programmable logic or circuitry (e.g., as encompassed within a general-purpose processor or other programmable processor) that is temporarily configured by software to perform certain operations. It will be appreciated that the decision to implement a hardware module mechanically, in dedicated and permanently configured circuitry, or in temporarily configured circuitry (e.g., configured by software) may be driven by cost and time considerations.

Accordingly, the term “hardware module” should be understood to encompass a tangible entity, be that an entity that is physically constructed, permanently configured (e.g., hardwired) or temporarily configured (e.g., programmed) to operate in a certain manner and/or to perform certain operations described herein. Considering embodiments in which hardware modules are temporarily configured (e.g., programmed), each of the hardware modules need not be configured or instantiated at any one instance in time. For example, where the hardware modules comprise a general-purpose processor configured using software, the general-purpose processor may be configured as respective different hardware modules at different times. Software may accordingly configure a processor, for example, to constitute a particular hardware module at one instance of time and to constitute a different hardware module at a different instance of time.

Hardware modules can provide information to, and receive information from, other hardware modules. Accordingly, the described hardware modules may be regarded as being communicatively coupled. Where multiple of such hardware modules exist contemporaneously, communications may be achieved through signal transmission (e.g., over appropriate circuits and buses) that connect the hardware modules. In embodiments in which multiple hardware modules are configured or instantiated at different times, communications between such hardware modules may be achieved, for example, through the storage and retrieval of information in memory structures to which the multiple hardware modules have access. For example, one hardware module may perform an operation and store the output of that operation in a memory device to which it is communicatively coupled. A further hardware module may then, at a later time, access the memory device to retrieve and process the stored output. Hardware modules may also initiate communications with input or output devices, and can operate on a resource (e.g., a collection of information).

The various operations of example methods described herein may be performed, at least partially, by one or more processors that are temporarily configured (e.g., by software) or permanently configured to perform the relevant operations. Whether temporarily or permanently configured, such processors may constitute processor-implemented modules that operate to perform one or more operations or functions. The modules referred to herein may, in some example embodiments, comprise processor-implemented modules.

Similarly, the methods described herein may be at least partially processor-implemented. For example, at least some of the operations of a method may be performed by one or processors or processor-implemented modules. The performance of certain of the operations may be distributed among the one or more processors, not only residing within a single machine, but deployed across a number of machines. In some example embodiments, the processor or processors may be located in a single location (e.g., within a home environment, an office environment or as a server farm), while in other embodiments the processors may be distributed across a number of locations.

The one or more processors may also operate to support performance of the relevant operations in a “cloud computing” environment or as a “software as a service” (SaaS). For example, at least some of the operations may be performed by a group of computers (as examples of machines including processors), with these operations being accessible via a network (e.g., the Internet) and via one or more appropriate interfaces (e.g., APIs).

Example embodiments may be implemented in digital electronic circuitry, or in computer hardware, firmware, software, or in combinations of them. Example embodiments may be implemented using a computer program product, for example, a computer program tangibly embodied in an information carrier, for example, in a machine-readable medium for execution by, or to control the operation of, data processing apparatus, for example, a programmable processor, a computer, or multiple computers.

A computer program can be written in any form of programming language, including compiled or interpreted languages, and it can be deployed in any form, including as a stand-alone program or as a module, subroutine, or other unit suitable for use in a computing environment. A computer program can be deployed to be executed on one computer or on multiple computers at one site or distributed across multiple sites and interconnected by a communication network.

In example embodiments, operations may be performed by one or more programmable processors executing a computer program to perform functions by operating on input data and generating output. Method operations can also be performed by, and apparatus of example embodiments may be implemented as, special purpose logic circuitry (e.g., a FPGA or an ASIC).

The computing system can include clients and servers. A client and server are generally remote from each other and typically interact through a communication network. The relationship of client and server arises by virtue of computer programs running on the respective computers and having a client-server relationship to each other. In embodiments deploying a programmable computing system, it will be appreciated that both hardware and software architectures require consideration. Specifically, it will be appreciated that the choice of whether to implement certain functionality in permanently configured hardware (e.g., an ASIC), in temporarily configured hardware (e.g., a combination of software and a programmable processor), or a combination of permanently and temporarily configured hardware may be a design choice. Below are set out hardware (e.g., machine) and software architectures that may be deployed, in various example embodiments.

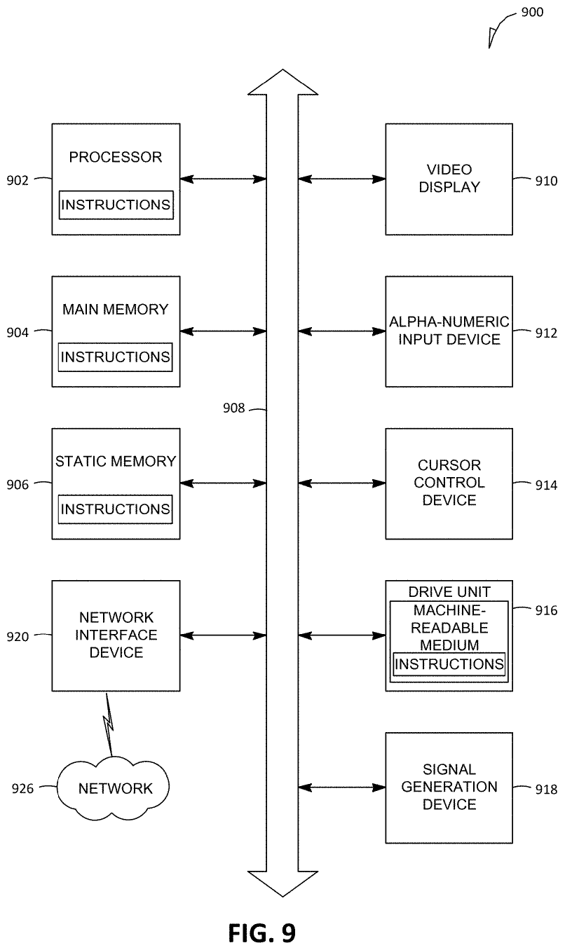

FIG. 9 is a block diagram of machine in the example form of a computer system 900 within which instructions, for causing the machine (e.g., client device 110, 115, 120, 125; server 135; database server(s) 140; database(s) 130) to perform any one or more of the methodologies discussed herein, may be executed. In alternative embodiments, the machine operates as a standalone device or may be connected (e.g., networked) to other machines. In a networked deployment, the machine may operate in the capacity of a server or a client machine in server-client network environment, or as a peer machine in a peer-to-peer (or distributed) network environment. The machine may be a personal computer (PC), a tablet PC, a set-top box (STB), a PDA, a cellular telephone, a web appliance, a network router, switch or bridge, or any machine capable of executing instructions (sequential or otherwise) that specify actions to be taken by that machine. Further, while only a single machine is illustrated, the term “machine” shall also be taken to include any collection of machines that individually or jointly execute a set (or multiple sets) of instructions to perform any one or more of the methodologies discussed herein.

The example computer system 900 includes a processor 902 (e.g., a central processing unit (CPU), a multi-core processor, and/or a graphics processing unit (GPU)), a main memory 904 and a static memory 906, which communicate with each other via a bus 908. The computer system 900 may further include a video display unit 910 (e.g., a liquid crystal display (LCD), a touch screen, or a cathode ray tube (CRT)). The computer system 900 also includes an alphanumeric input device 912 (e.g., a physical or virtual keyboard), a user interface (UI) navigation device 914 (e.g., a mouse), a disk drive unit 916, a signal generation device 918 (e.g., a speaker) and a network interface device 920.

The disk drive unit 916 includes a machine-readable medium 922 on which is stored one or more sets of instructions and data structures (e.g., software) 924 embodying or used by any one or more of the methodologies or functions described herein. The instructions 924 may also reside, completely or at least partially, within the main memory 904, static memory 906, and/or within the processor 902 during execution thereof by the computer system 900, the main memory 904 and the processor 902 also constituting machine-readable media.

While the machine-readable medium 922 is shown in an example embodiment to be a single medium, the term “machine-readable medium” may include a single medium or multiple media (e.g., a centralized or distributed database, and/or associated caches and servers) that store the one or more instructions or data structures. The term “machine-readable medium” shall also be taken to include any tangible medium that is capable of storing, encoding or carrying instructions for execution by the machine and that cause the machine to perform any one or more of the methodologies of the present invention, or that is capable of storing, encoding or carrying data structures used by or associated with such instructions. The term “machine-readable medium” shall accordingly be taken to include, but not be limited to, solid-state memories, and optical and magnetic media. Specific examples of machine-readable media include non-volatile memory, including by way of example, semiconductor memory devices (e.g., Erasable Programmable Read-Only Memory (EPROM), Electrically Erasable Programmable Read-Only Memory (EEPROM)) and flash memory devices; magnetic disks such as internal hard disks and removable disks; magneto-optical disks; and CD-ROM and DVD-ROM disks.

The instructions 924 may further be transmitted or received over a communications network 926 using a transmission medium. The instructions 924 may be transmitted using the network interface device 920 and any one of a number of well-known transfer protocols (e.g., HTTP). Examples of communication networks include a LAN, a WAN, the Internet, mobile telephone networks, Plain Old Telephone (POTS) networks, and wireless data networks (e.g., WiFi and WiMax networks). The term “transmission medium” shall be taken to include any intangible medium that is capable of storing, encoding or carrying instructions for execution by the machine, and includes digital or analog communications signals or other intangible media to facilitate communication of such software.

### EXAMPLES

Data and Methods

In an example, ICU stay records were analyzed to determine early prediction factors for development of septic shock. The publicly accessible records were from the Medical Information Mart for Intensive Care (MIMIC III) database, which contains ˜40,000 ICU stay records and is a suitable resource for research and discovery of predictive factors for various outcomes. The MIMIC-III Clinical Database contains health records on Intensive Care Unit admissions from 2001 to 2012 at the Beth Israel Deaconess center. This dataset is publicly available and contains a wide range of medical data including laboratory test results, vital signs, diagnosis and procedure codes etc. The data was imported from CareVue clinical information system for admissions between 2001 and 2008 and Meta Vision (provided by iMDSoft) for admissions between 2008 and 2012. The local copy of the database was hosted on a Hadoop cluster and was queried using Hive. R language was used for the data processing and statistical analysis. Visit level data including ADT, chart data, lab results, microbiology results, diagnosis code and procedure code data was extracted from the database MIMIC-III database.

Criterion for Identification of Patients that Have Progressed into Septic Shock

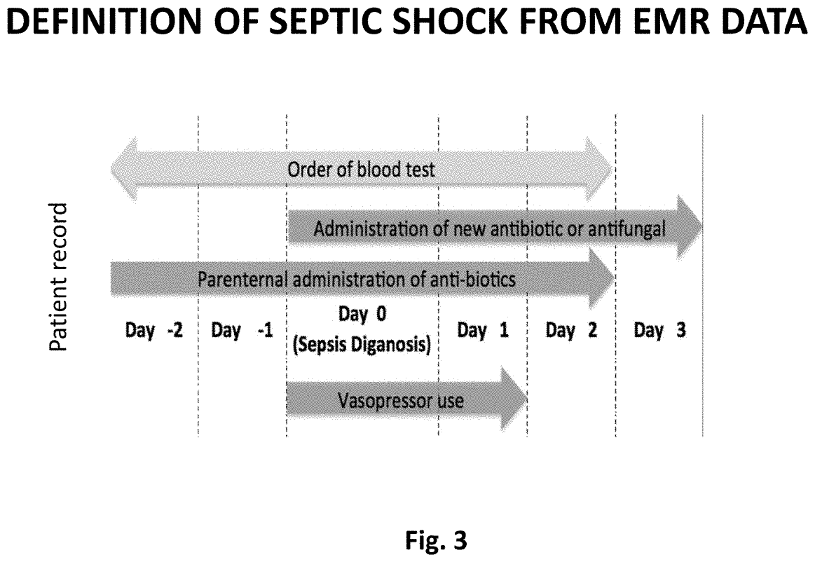

A working definition for digital diagnosis of septic shock is described herein. The definition provided here is used as the “gold standard” for (i) building a novel predictive method for septic shock (ii) evaluating performance of a novel predictive method for septic shock (iii) evaluating performance of widely used scoring systems for sepsis. The criterion for identifying patients in septic shock from electronic medical record data was based on earlier work by Kadri et al. (Kadri S S, Rhee C, Strich J R, et al. Estimating ten-year trends in septic shock incidence and mortality in United States Academic Medical Centers using clinical data. Chest. 2017; 151(2):278-285), which is incorporated by reference herein in its entirety. Occurrence of septic shock in each patient was identified based on two conditions: (i) Presence of an indicator of shock (vasopressor use) and (ii) At least one indicator of presumed infection: blood culture test, administration of antibiotics or antifungals. Day 0 is considered to be the day of onset of septic shock. FIG. 3 graphically depicts the time lines and the clinical criterion used to identify progression to septic shock in a patient during their stay in the ICU as identified based on electronic medical record data. A patient is considered to have gone into septic shock on day 0 if the following conditions are met: (i) Relevant vasopressor use occurs on day 0 and day 1. Vasopressor drugs that were considered are: Norepinephrine, Epinephrine, Vasopressin, Dopamine, and Phenylephrine. Vasopressors were identified from the input events and chart events tables in the MIMIC III database, (ii) Relevant blood culture orders were made from day −2 to day 2 relative to the onset of septic shock. Blood culture orders were identified from the microbiology events table from the database, (iii) Administration of a new antibiotic or antifungal from day 0 to day 3 is considered relevant to identification of onset of septic shock. Similarly, parenteral administration of antibiotic between day −2 and day 2 is also considered relevant. Drug administration was identified from the input events table based on National Drug Codes (NDCs). NDCs from the MIMIC III database were mapped to the complete NDC directory as listed by the Food and Drug Administration. (C. for D. E. and Research, “Drug Approvals and Databases—National Drug Code Directory.” [Online]. Available at www.fda.gov/dmgs/informationondrugs/ucm142438.htm. [Accessed: 8 Mar. 2019].) The drug class categories as defined in the NDC directory were used to identify 2,704 antibacterial and 560 antifungal drugs. Based on route of administration information from the NDC directory, 1,252 antibacterial and 226 antifungal drugs were identified to characterize parenteral administration of antimicrobials.

Patient Cohort Selection

A cohort of “Septic shock patients” or cases was built by applying the definition for digital diagnosis of septic shock as described previously. A matching control cohort was identified by randomly sampling from all admissions in the database but not including patients that have been identified as progressing into septic shock. For each patient in the control cohort, a time point during their ICU stay was chosen for notional diagnosis of septic shock. The time points for notional diagnosis of septic shock in the control patient population were chosen as follows: (i) the number of hours of hospital stay prior to diagnosis of septic shock was identified for each patient in the septic shock cohort, and (ii) the time point of notional septic shock for each patient in the control cohort was sampled from the time points calculated from the septic shock cohort.

Data Processing for Construction of Bayesian Networks

For patients in the septic shock and control cohort, data relevant to lab events, specifically the lab test item ID and the results from manual interpretation were extracted from the MIMIC III database for all visits. The extracted lab data was inherently discrete with the following levels: normal, abnormal, delta (a large or sudden change of a lab result from the previous test result) and missing data. If a lab measurement was not made for the time window under consideration, the lab measurement was interpreted as “not measured”. ICD9 diagnosis and procedure codes for both cohorts were extracted from the MIMIC III dataset. The large number of ICD9 codes was reduced using the Clinical Classification Software (CCS) algorithms from the Agency for Healthcare Research and Quality. The CCS dataset allows for group similar diagnostic and procedure codes to increase the density of the data and improve interpretability of the codes.

A dataset representing snapshots of the medical record of the patients 24 hours prior to the diagnosis of septic shock was created. For lab data, to increase the completeness of the patient profiles, all lab result data between 24 and 30 hours prior to diagnosis of septic shock was considered. For diagnosis and procedure codes, if the code was observed any time prior to 24 hours preceding septic shock diagnosis, it was assigned as “present”. If the diagnosis or procedure code was not observed any time prior to the time point, it was assigned as “absent”.

Model Building Using bAIcis Analytical Platform

An integrated dataset containing lab data, diagnosis, procedure and observation of septic shock was created containing all patients in both the septic shock and control. A Bayesian network containing inferred cause and effect relationships between variables in the dataset was built using the bAIcis Analytical Platform of Berg, LLC. The method employed for creation of the causal Bayesian networks has been described in previous studies (V. Vemulapalli et al, “Non-obvious correlations to disease management unraveled by Bayesian artificial intelligence analyses of CMS data,” Artificial Intelligence in Medicine, vol. 74, pp. 1-8, November 2016; M. Huang, C. R. Yee, V. P. Sukhatme, N. R. Narain, V. R. Akmaev, and V. Vemulapalli, “Identification of Novel Risk Factors for Hospital Admission after Colonoscopy Exam by Analyzing Population Wide Data with Artificial Intelligence Technology bAIcis®,” Proceedings of The 22nd World Multi-Conference on Systemics, Cybernetics and Informatics (WMSCI2018), vol. Volume III, pp. 130-133, 2018.), which are incorporate by reference herein in their entireties. Additional information regarding creation of Bayesian networks may be found in commonly owned U.S. Patent Application Publication No. 2016/0171383, which is incorporated by reference herein in its entirety. Demographic information was considered to be fixed information regarding the patients. Therefore, in the causal network, no other variables were permitted to drive changes in demographic characteristics of the patients. Due to temporal nature of the septic shock diagnosis in relation to other data elements, the diagnosis of septic shock was not permitted to causally drive changes in other variables in the dataset. An ensemble of 500 causal networks was built and the results were summarized as a single representative network. The edges (edges link variables/nodes in network) were weighted based on the frequency of observation in the ensemble of networks. The summarized causal network was then filtered to retain only interactions that were observed with a frequency of 0.4 or greater. A sub-network was selected from the summarized network to include risk factors that drive progression into septic shock. A regression model was built using factors from the sub-network around the outcome of septic shock. Multiple thresholds were selected to obtain the most desirable model performance in a clinical application. Factors included in the regression model were manually selected from the subnetwork based on their power to predict septic shock.

Risk Factor Selection for Predictive Model

All first and second degree neighbors of septic shock diagnosis from the cause-and-effect networks were considered for final model selection. For each Bayesian network, step-wise forward selection was performed to identify neighbors that were most predictive of septic shock. Based on manual analysis of the selected factors, lab results and other indicators that were most predictive of sepsis were identified. Threshold for the general linear model (GLM) for predicting septic shock was selected to maximize sensitivity and specificity. All variables considered were discrete variables as described earlier in the methods section. A de-novo predictive model was built based on lab values that were identified through this method.

Two different predictive models were developed, as explained below in Example 1 and Example 2.

### Example 1—Model Including Lab Results and Diagnostic Codes

In the analysis of Example 1, based on the definition of septic shock employed 872 admissions (case) out of a total of 58,976 admissions were identified as having had a diagnosis of sepsis and made up the patent cohort for sepsis as shown in Table 1. 1,768 control admissions were randomly selected where no diagnosis of septic shock was possible based on criterion used. All analysis was performed in the basis of admissions.

TABLE 1

| Patient | Number of | Number of |
|---|---|---|
| cohort | Patients | Admissions |
| All | 46,520 | 58,976 |
| Sepsis | 872 (1.87%) | 884 (1.50%) |
| Control | 8,293 | 8,772 |

Data was generated for both cohorts to create a snapshot of the patient 24 hours prior to sepsis. To enable more complete patient records, all data 6 hours prior to the time point was used. Demographics, lab data, diagnosis and procedure data was used in building Bayesian networks.

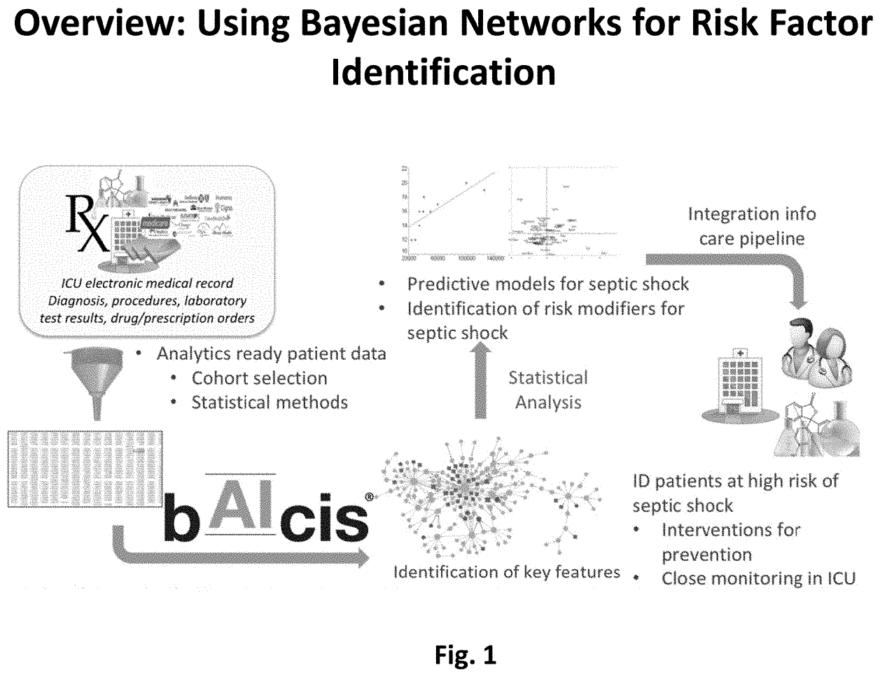

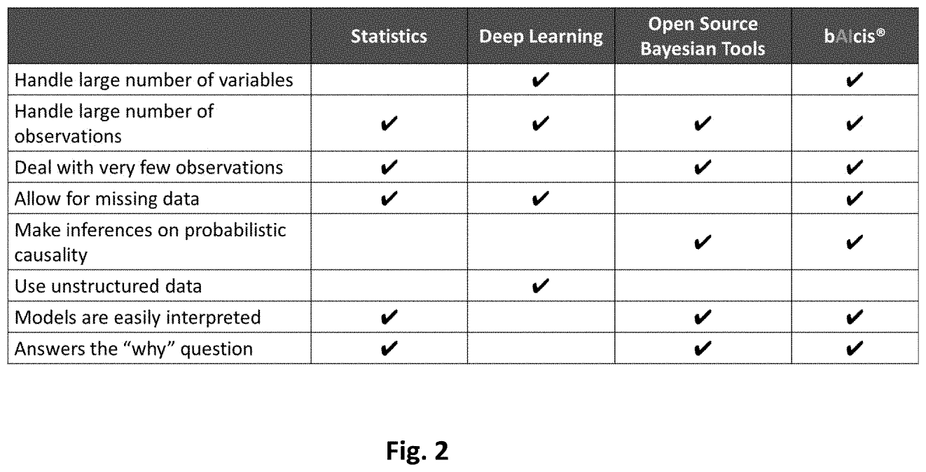

All data types extracted from the database were discretized and processed for Bayesian network analysis. The possible discrete values for the lab values were “not measured”, “normal” and “abnormal”. Advantages of using Bayesian networks and high-level workflow are presented in FIG. 1 and FIG. 2. For this example, separate datasets were created representing the clinical status of the patient cohorts at 6, 8, 10, 12 and 24 hours before diagnosis of septic shock. Because measurement intervals of the data varied widely by both patient and data type, missing data was imputed based on the carry-forward method when possible, for example, 3 and 6 hour windows preceding timepoints mentioned earlier were used. Separate Bayesian networks were built for each time point.

As graphically depicted in FIG. 1, various data elements from the MIMIC III database were processed for statistical analysis. De-novo, data-derived Bayesian cause and effect networks were built by using the bAIcis® analysis platform. Key features were validated and encoded as predictive algorithms through use of standard statistical approaches such as regression models. FIG. 1 depicts determining a de-novo data-driven Bayesian cause and effect network based on ICU electronic medical record data, such as diagnosis, procedures, laboratory test results, drug prescription orders, etc., and the identification of key features from the Bayesian network for identifying risk modifiers for septic shock and for of development of predictive models of septic shock. FIG. 2 lists features of the bAIcis® system that enable it to overcome many limitations of classical statistical analysis, deep learning methods and open-source Bayesian tools in some embodiments. Most relevant to this work are the ability to generate transparent/explainable predictive models, and, in this context, it enables entirely data-driven, unbiased identification of potential key factors that drive risk of septic shock in patients in the ICU. The resulting Bayesian networks built using the bAIcis network module represented up to 300 variables from the database and 550 interactions between the variables. The variables most relevant to prediction of septic shock were identified based on connectivity to sepsis. Various features identified as risk factors for septic shock include lab measures, ICD9 diagnosis and ICD9 procedure codes. Although separated networks were analyzed for 6, 8, 100, 12 and 24 hours before diagnosis of septic shock, results corresponding to 24 hours prior to onset of sepsis are described herein because early detection is likely to result in better outcomes for patients ,

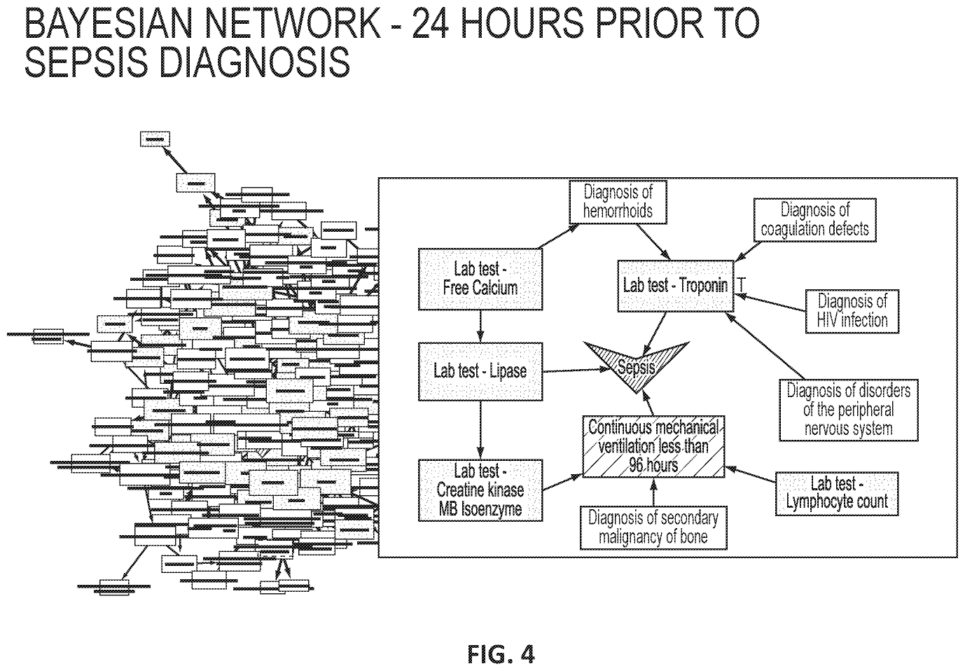

FIG. 4 depicts a full Bayesian causal relationship network of inferred causal relationships between various clinical variables at 24 hours prior to the diagnosis of sepsis. The smaller inset subnetwork of FIG. 4 shows clinical variables connected to sepsis. In the networks, V-shaped nodes are clinical outcomes of interest (death, sepsis), dark gray rectangles are lab measurements, dark gray rectangles with broken outlines are procedures, and light gray rectangles are diagnoses. Variables connected to sepsis were analyzed for differentiation between the septic shock cohort and the control cohort and to determine if they were confounded. Based on the analysis, specific factors predictive of septic shock were identified and a detailed statistical model was constructed.

Specific factors predictive of septic shock included lab test results indicating whether a measured level for mean corpuscular hemoglobin fell outside a normal range, and lab test results indicating whether a measured level of whole blood potassium fell outside a normal range. Another specific factor predictive of septic shock was whether the patient was diagnosed with a disease or disorder falling in the following International Classification of Disease 9 Clinically Modified (ICD-9-CM) Codes: 2760 (hyperosmolality and/or hypernatremia), 2762 (acidosis), 2763 (alkalosis), 2764 (mixed acid-base balance disorder), 2766 (fluid overload), 27669 (fluid overload not elsewhere classified), 2769 (Electrolyte and fluid disorders not elsewhere classified), and 9951 (Angioneurotic edema not elsewhere classified).

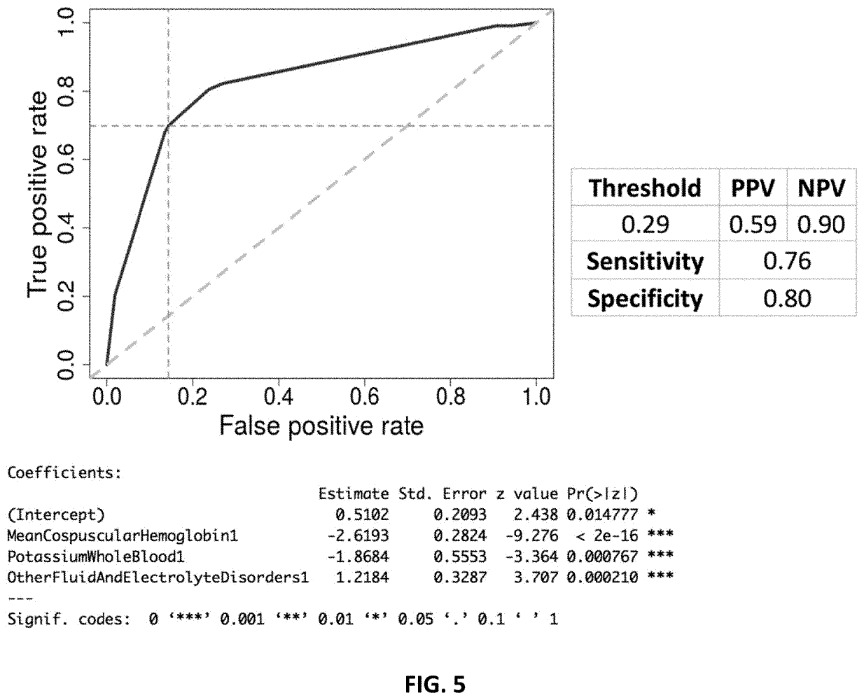

A logistic regression model was created based on these three factors/variables. The model input regarding mean corpuscular hemoglobin of the patient was normal or abnormal. For example, if the level of the mean corpuscular hemoglobin fell in the normal range for a patient, the corresponding first factor/variable was assigned a value of zero and if the level of the mean corpuscular hemoglobin fell outside the normal range, the first factor/variable was assigned a value of 1. The model input regarding a level of total blood potassium for the patient was normal or abnormal. For example, if the level of the total blood potassium fell in the normal range for a patient, the corresponding second factor/variable was assigned a value of zero and if the level of the total blood potassium fell outside the normal range the corresponding second factor/variable was assigned a value 1. The model input regarding whether the patient had been diagnosed with any of the specified disorders was yes or no. For example, if the patient was diagnosed with any of the specified disorders, the corresponding third factor/variable was assigned a 1, otherwise it was assigned a 0. The details of the regression model and the results of the regression model are listed in Table 2 below and presented in FIG. 5. The coefficients corresponding to each factor/variable and various model parameters are shown in the table below, where the column “Estimate” is estimate of the coefficient, the column “Std. Error” is standard error on the estimate of the coefficient, the “z value” column is the estimate of the coefficient divided by the standard error, and the Pr(>|z|) is the P-value. The smaller P-value, the more likely that the variable/factor is an important predictor.

TABLE 2
Coefficients:

| | Estimate | Std. Error | z value | Pr(>\|z\|) | |
|---|---|---|---|---|---|
| (Intercept) | 0.5102 | 0.2093 | 2.438 | 0.014777 | * |
| MeanCospuscularHemoglobin1 | −2.6193 | 0.2824 | −9.276 | <2e−16 | *** |
| PotassiumWholeBlood1 | −1.8684 | 0.5553 | −3.364 | 0.000767 | *** |
| OtherFluidAndElectrolyteDisorders1 | 1.2184 | 0.3287 | 3.707 | 0.000210 | *** |

Signif. codes: 0
‘***’ 0.001
‘**’ 0.01
‘*’ 0.05
‘.’ 0.1
‘’ 1

A detailed model and specific factors that were found to be predictive of septic shock are shown in FIG. 5. A threshold value that maximized both sensitivity and specificity was selected to distinguish between patients that experienced septic shock from the control patient cohort. The model and selected threshold had a sensitivity of 0.76 and a specificity of 0.80. In other embodiments, the threshold may be modified to adjust a positive predictive value (PPV), a negative predictive value (NPV), a selectivity or a specificity.

### Example 2—Model Including Lab Results and Whether Values were Measured

A second analysis was conducted on the MIMIC III data for building a predictive model for septic shock.

### Patient Cohorts and Novel Predictive Model for Septic Shock

In the analysis of Example 2, 872 septic shock patients 8,293 and control patients were identified from the MIMIC III database. Septic shock patients were identified using the definition for digital diagnosis as described in the methods section. The control cohort only contains patients that did not have a digital diagnosis of septic shock during their stay at the ICU. Details of the patient populations are shown in Table 3 and Table 4 below.

TABLE 3

Number of patients

| Patient cohort | Complete dataset | 24 hour window |
|---|---|---|
| All | 46,520 | 534 |
| Septic shock | 872 (1.87%) | 142 (26.6%) |
| Control | 8,293 | 391 |

TABLE 4

| Patient | | Complete Cohort | | | | 24 hour Cohort | | | |
|---|---|---|---|---|---|---|---|---|---|
| Characteristics | Type | Control | | Septic shock | | Control | | Septic shock | |
| Gender | M | 3919 | (44.68%) | 459 | (54.71%) | 211 | (53.96%) | 79 | (55.63%) |
| | F | 4853 | (55.32%) | 380 | (45.29%) | 180 | (46.04%) | 63 | (44.37%) |
| Age (years) | <18 | 1200 | (13.68%) | 28 | (3.34%) | 0 | (0%) | 0 | (0%) |
| | 18-30 | 380 | (4.33%) | 17 | (2.03%) | 17 | (4.35%) | 4 | (2.82%) |
| | 31-50 | 1350 | (15.39%) | 102 | (12.16%) | 78 | (19.95%) | 23 | (16.2%) |
| | 51-60 | 1291 | (14.72%) | 142 | (16.92%) | 65 | (16.62%) | 23 | (16.2%) |
| | 61-70 | 1595 | (18.18%) | 141 | (16.81%) | 66 | (16.88%) | 20 | (14.08%) |
| | 71-SO | 1521 | (17.34%) | 173 | (20.62%) | 81 | (20.72%) | 36 | (25.35%) |
| | 81-89 | 1047 | (11.94%) | 171 | (20.38%) | 52 | (13.3%) | 33 | (23.24%) |
| | 90+ | 388 | (4.42%) | 65 | (7.75%) | 32 | (8.18%) | 3 | (2.11%) |
| Length of stay | 0 | 95 | (1.08%) | 116 | (13.83%) | 0 | (0%) | 0 | (0%) |
| (days) | 1-5 | 3584 | (40.86%) | 546 | (65.08%) | 141 | (36.06%) | 75 | (52.82%) |
| | 6-10 | 2521 | (28.74%) | 101 | (12.04%) | 135 | (34.53%) | 37 | (26.06%) |
| | 11-15 | 1063 | (12.12%) | 33 | (3.93%) | 51 | (13.04%) | 11 | (7.75%) |
| | 16+ | 33 | (0.38%) | 2 | (0.24%) | 64 | (16.37%) | 19 | (13.38%) |
| Died during | Yes | 687 | (7.83%) | 835 | (99.52%) | 29 | (7.42%) | 142 | (100%) |
| ICU stay | No | 8085 | (92.17%) | 4 | (0.48%) | 362 | (92.58%) | 0 | (0%) |

As shown in Table 4, the patient cohorts have differences in the demographic characteristics. For example, there is a difference in age distribution of patients in the 2 cohorts. It is already known that patient age increases the risk of sepsis. In line with this expectation Table 4 shows that the control population tends to be younger. A chi-square test was performed to compare the patient characteristics between the control and septic shock populations. The null hypothesis that both control and sepsis populations are similar was rejected for all 4 characteristics (p-value<0.01). Septic shock patients tend to be older, have a higher proportion of males and have higher death rates during ICU stay. Diagnosis of septic shock is associated with significantly higher mortality rates while in the ICU (Relative risk of death in septic shock patients=2.09) as has been documented in previous studies (see G. S. Martin, “Sepsis, severe sepsis and septic shock: changes in incidence, pathogens and outcomes,” Expert Rev Anti Infect Ther, vol. 10, no. 6, pp. 701-706, June 2012; C. Brun-Buisson, F. Doyon, and J. Carlet, “Bacteremia and severe sepsis in adults: a multicenter prospective survey in ICUs and wards of 24 hospitals. French Bacteremia-Sepsis Study Group,” Am. J. Respir. Crit. CareMed., vol. 154, no. 3 Pt 1, pp. 617-624, September 1996).

For each patient in the control cohort, a time point for notional diagnosis of sepsis was chosen as described in the methods section. A time point for notional diagnosis of septic shock was chosen in order to temporally align patients from the septic shock and control cohorts. Patients in the control cohort do not have a diagnosis of septic shock at the time of notional septic shock, however, relevant lab and diagnosis code data for the control cohort patients was determined based on the time of notional septic shock. Because it has been shown in several studies that even small delays in diagnosis of sepsis can lead to significantly impacts on outcomes, the model was targeted to predict septic shock 24 hour ahead of time (see A. Kumar et al., “Duration of hypotension before initiation of effective antimicrobial therapy is the critical determinant of survival in human septic shock,” Crit. Care Med., vol. 34, no. 6, pp. 1589-1596, June 2006; R. Ferrer et al, “Empiric antibiotic treatment reduces mortality in severe sepsis and septic shock from the first hour: results from a guideline-based performance improvement program,” Crit. Care Med., vol. 42, no. 8, pp. 1749-1755, August 2014; B. B. Whiles, A. S. Deis, and S. Q. Simpson, “Increased Time to Initial Antimicrobial Administration Is Associated With Progression to Septic Shock in Severe Sepsis Patients,” Crit. Care Med., vol. 45, no. 4, pp. 623-629, April 2017). Patient data from 24-30 hours prior to diagnosis of septic shock was processed and organized for statistical analysis using Bayesian networks.

Data from both cohorts was merged and Bayesian cause-and-effect networks were inferred from this dataset. 533 patients were included in the Bayesian network analysis based on availability of data. This includes 391 patients in the control cohort and 186 patients in the septic shock cohort. The dataset consisted of interpretations of 48 lab tests, 126 ICD9 diagnosis code groups, 4 procedure code groups, patient sex, patient age, death during ICU stay and digital diagnosis status of septic shock. In this cohort of 533 patients, a large proportion of the patients that died during the ICU stay had a digital diagnosis of septic shock (43% percent) comparable to published accounts of causes of ICU mortality (see D. C. Angus and R. S. Wax, “Epidemiology of sepsis: an update,” Crit. Care Med., vol. 29, no. 7 Suppl, pp. S109-116, July 2001; A. Braber and A. R. van Zanten, “Unravelling post-ICU mortality: predictors and causes of death,” European Journal of Anesthesiology (EJA), vol. 27, no. 5, p. 486, May 2010).

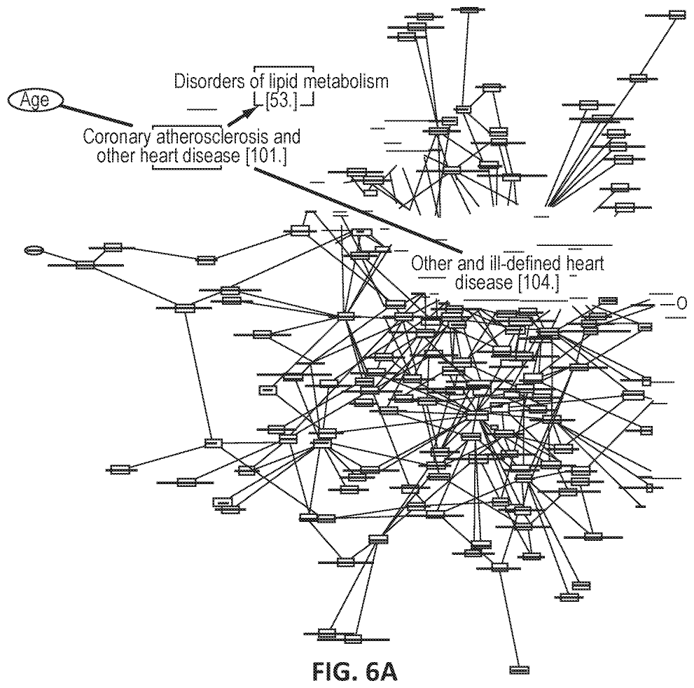

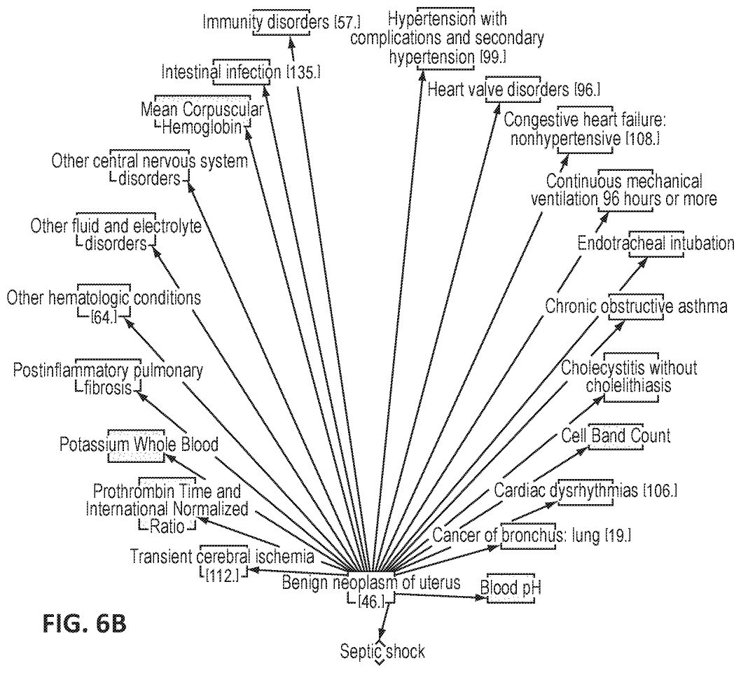

FIG. 6A shows the complete generated Bayesian cause-and-effect network and some sample subnetworks. Specifically, FIG. 6A shows the complete summary network from bAIcis® with a zoomed-in view of a portion of the complete network including age superimposed on the complete network. The network, after filtering out low-frequency edges, consisted of 181 nodes (corresponding to relationships) connected by 353 edges (corresponding to features such as variables or parameters). When two data features are connected by an edge (direct relationship) in the network, it should be interpreted as one feature (demographics, diagnosis, procedure or lab result) probabilistically leading to another feature (new diagnosis, procedure being performed or specific lab test status) in a patient. For example, from the inset in FIG. 6A, it can be inferred that patient age trends with a high chance of diagnosis of ‘coronary atherosclerosis and other heart disease’. This inference is in line with the knowledge that the risk of atherosclerosis and heart disease goes up with age. As a second example, from the inset panel ‘disorders of lipid metabolism’ which includes codes for high cholesterol levels is linked to ‘coronary atherosclerosis and heart disease’ as expected. A sub-network around septic shock is shown in FIG. 6B. Specifically, FIG. 6B is a subnetwork including the first and second degree neighbors of septic shock. Several diagnosis code groups and lab tests are linked to septic shock. In FIGS. 6A and 6B, the gray ellipses are patient demographics, (e.g., age) the gray diamonds are outcomes (e.g., death during ICU stay, septic shock), and the gray rectangles are diagnosis and procedure codes and lab tests.

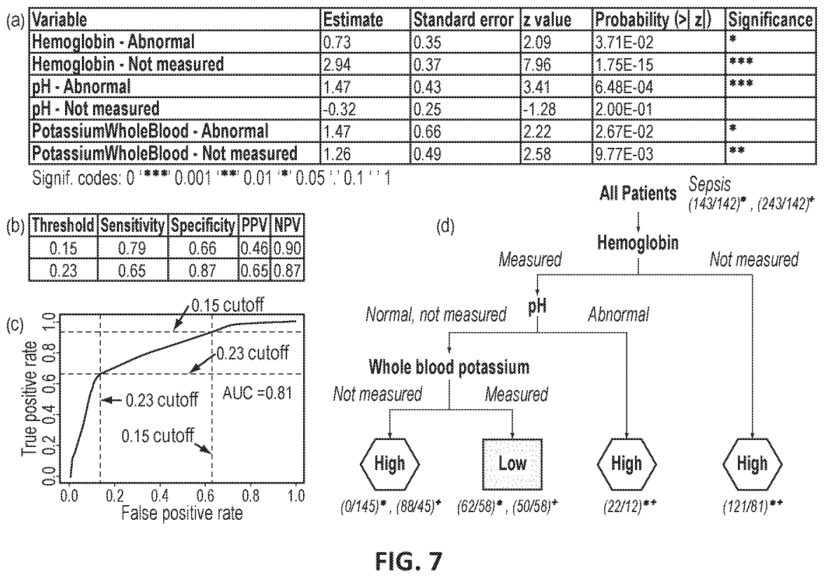

The regression model that was selected from the selected sub-network (from FIG. 6B) in Example 2 is shown in FIG. 7. FIG. 7 also shows details of the predictive model that was built and its performance characteristics. The model classifies patients in high or low risk categories for sepsis based on the status of several lab tests. The lab tests in the predictive model (Hemoglobin levels, blood pH and whole blood potassium levels) were assigned possible values of: not measured and measured (normal or abnormal). A tree representation of the regression model is also shown in FIG. 7 to enable easier interpretation of the model.

Based on its performance characteristics, this model can be used to screen for patients that are at high-risk of sepsis leading to earlier diagnosis of sepsis. Earlier detection of sepsis is known to result in better outcomes; therefore use of the model or systems or modules based on the model can facilitate better allocation of resources to identify patients with sepsis early in the course of the disease. Despite the relatively small number of patients that develop septic shock, this algorithm has moderately high positive predictive value and high negative predictive value.

Panel (a) shows the details of the regression model for predicting patients' risk of progressing into septic shock 24 hours prior to diagnosis of septic shock. Table 5 below includes the coefficients of the regression model.

TABLE 5

| | | Standard | z | Probability | |
|---|---|---|---|---|---|
| (a) Variable | Estimate | error | value | (>\|z\|) | Significance |
| Hemoglobin - Abnormal | 0.73 | 0.35 | 2.09 | 3.71E−02 | * |
| Hemoglobin - Not measured | 2.94 | 0.37 | 7.96 | 1.75E−15 | *** |
| pH - Abnormal | 1.47 | 0.43 | 3.41 | 6.48E−04 | *** |
| pH - Not measured | −0.32 | 0.25 | −1.28 | 2.00E−01 | |
| PotassiumWholeBlood - | 1.47 | 0.66 | 2.22 | 2.67E−02 | * |
| Abnormal | | | | | |
| PotassiumWholeBlood - | 1.26 | 0.49 | 2.58 | 9.77E−03 | ** |
| Not measured | | | | | |

Signif. codes: 0
‘***’ 0.001
‘**’ 0,01
‘*’ 0.05
‘.’ 0.1
‘’ 1
All patients Sepsis (143/142)*, (243/142)*

Panel (b) shows two different model cutoffs that were selected for assessing model performance. Two different thresholds were selected to allow for different false negative and true positive rates. The thresholds are: 0.23 (*) and 0.15(+). Predictions are made to classify patients as “high” risk and “low” risk. For each prediction, the following information is presented: (number predicted/actual numbers based on assessed septic shock risk).

Panel (c) shows the ROC curve for model with both the 0.15 cutoff and the 0.23 cutoff indicated with labeled dashed lines. The AUC curve was built by interpolating between points measured in the dataset because predictive data was discrete. Panel (d) shows a decision tree that represents the regression model used to identify patients at high risk of sepsis. This model could be incorporated into a screening tool, screening system, patient care system, clinical decision support system for patient care, or other suitable system or module to identify patients at high risk of developing septic shock. Incorporation of the model would enable targeted close monitoring of identified high risk patients to increase the chances of early detection and thereby optimizing usage of clinical resources.

Comparison to Current Tools for Evaluation for Septic Shock

A few different scoring metrics have been created for clinical use to track sepsis in patients in ICU. One of the most well-known scores to assess sepsis is the SOFA score (Sepsis-related Organ Failure Assessment). It was introduced in 1996 by Vincent et al. (see J. L. Vincent et al, “The SOFA (Sepsis-related Organ Failure Assessment) score to describe organ dysfunction/failure. On behalf of the Working Group on Sepsis-Related Problems of the European Society of Intensive Care Medicine,” Intensive Care Med, vol. 22, no. 7, pp. 707-710, July 1996). A variant of the SOFA score, qSOFA is primarily used for mortality prediction, but not to clinical track sepsis. MEWS ((Modified early warning score) has been used as a tool for screening for sepsis and to identify patients at risk for clinical deterioration (see J. K. Roney, B. E. Whitley, J. C. Maples, L. S. Futrell, K. A. Stunkard, and J. D. Long, “Modified early warning scoring (MEWS): evaluating the evidence for tool inclusion of sepsis screening criteria and impact on mortality and failure to rescue,” J Clin Nurs, vol. 24, no. 23-24, pp. 3343-3354, December 2015). SIRS (Systemic inflammatory response syndrome) scores are also used to assess patient sepsis status (see R. C. Bone et al, “Definitions for sepsis and organ failure and guidelines for the use of innovative therapies in sepsis. The ACCP/SCCM Consensus Conference Committee. American College of Chest Physicians/Society of Critical Care Medicine,” Chest, vol. 101, no. 6, pp. 1644-1655, June 1992; M. M. Levy et al, “2001 SCCM/ESICM/ACCP/ATS/SIS International Sepsis Definitions Conference,” Crit. Care Med., vol. 31, no. 4, pp. 1250-1256, April 2003).

SOFA, qSOFA, MEWS and SIRS were calculated for comparison. SOFA scores were calculated based on published guidelines using data from MIMIC III (see A. E. Jones, S. Trzeciak, and J. A. Kline, “The Sequential Organ Failure Assessment score for predicting outcome in patients with severe sepsis and evidence of hypoperfusion at the time of emergency department presentation,” Crit. Care Med., vol. 37, no. 5, pp. 1649-1654, May 2009). Data from the chart events, lab events, and medication and diagnosis tables were used. Relevant item IDs corresponding to clinical measures of interest, such as coagulations were identified though text searches and manual matching. Six categories (GCS, liver function, coagulation, renal function, respiratory function and cardiovascular function) were evaluated and the total SOFA score was calculated. Changes in SOFA score could not be estimated from the data due to lack of resolution in the dataset. Alternatively a threshold of 4 was chosen to assign patients to the ‘high risk of septic shock’ group. If all measures were unavailable or could not be calculated from the data and the SOFA score was less than the threshold, the SOFA score was set to ‘NA’ to indicate it could not be calculated. For all remaining patients an assignment of ‘low risk of septic shock’ was made. qSOFA score was calculated by accounting for mental status, respiratory rate and blood pressure as described by Seymour et al., which is incorporated by reference in its entirety (see C. W. Seymour et al, “Assessment of Clinical Criteria for Sepsis: For the Third International Consensus Definitions for Sepsis and Septic Shock (Sepsis-3),” JAMA, vol. 315, no. 8, pp. 762-774, February 2016). If the qSOFA score was 2 or higher, an assignment of ‘high risk of septic shock’ was made. If the qSOFA score was 1 with all 3 measures available in the dataset, an assignment of ‘low risk of septic shock’ was made. For the remaining patients, qSOFA based assignment of septic shock was set to ‘NA’.

The systemic inflammatory response syndrome (SIRS) score was calculated based on 4 criterion: body temperature, heart rate, respiratory rate, and white blood cell counts (see G. S. Martin, “Sepsis, severe sepsis and septic shock: changes in incidence, pathogens and outcomes,” Expert Rev Anti Infect Ther, vol. 10, no. 6, pp. 701-706, June 2012). Patients whose scores added up to 2 or greater were considered positive for SIRS (‘high risk of septic shock’), while other patients where the score was calculated were negative for SIRS (low risk of septic shock’). Modified early warning score (MEWS) (see C. P. Subbe, M. Kruger, P. Rutherford, and L. Gemmel, “Validation of a modified Early Warning Score in medical admissions,” QJM, vol. 94, no. 10, pp. 521-526, October 2001) was calculated based on systolic blood pressure, heart rate, respiratory rate, temperature, as well as the alert, voice, pain, unresponsive (AVPU) scores. The AVPU scores (see C. A. Kelly, A. Upex, and D. N. Bateman, “Comparison of consciousness level assessment in the poisoned patient using the alert/verbal/painful/unresponsive scale and the Glasgow Coma Scale,” Ann Emerg Med, vol. 44, no. 2, pp. 108-113, August 2004), which are an approximation of the Glasgow Coma Scores, were not directly available in the MIMIC III database and hence were estimated. The GCS scores were used in place of AVPU scores based on work by Kyriacos et al. (see U. Kyriacos, J. Jelsma, M. James, and S. Jordan, “Monitoring vital signs: development of a modified early warning scoring (MEWS) system for general wards in a developing country,” PLoS ONE, vol. 9, no. 1, p. e87073, 2014). A MEWS score of 2 or greater was used for an assignment of ‘high risk of septic shock’ while a combination of availability of all data and score of less than 2 was used for assignment of ‘low risk of septic shock’. All other patients were assigned to ‘NA’ category based on MEWS scores.

Performance of SOFA, qSOFA, SIRS and MEWS on this dataset and performance of these scores as augmented using the lab values identified for use in the regression model above is shown in Table 6.

Model performance evaluation was only conducted for patients for whom both the score calculation and data availability for 24 hour time point were available. It was observed that while the SOFA score was more difficult to calculate, the measures to calculate the score were more readily available (279 patients in cohort). Cohorts for other scores were smaller due to more of the required data for score being missing. The 4 standard models (SOFA, qSOFA etc.) were augmented with the new factors (Hemoglobin, pH and whole blood potassium) that were discovered in this work. Youden's index was used for threshold selection in the score only models. Comparison of the augmented models to the score only models was done by fixing either sensitivity or specificity of the augmented model to that of the score only models.

TABLE 6

| | | | Negative | Positive | | |
|---|---|---|---|---|---|---|
| | Sample | | predictive IT | predictive | | |
| Model | size | Match by | value | value | Sensitivity | Specificity |
| qSOFA | 46 | — | 0.55 | 0.54 | 0.55 | 0.54 |
| Augmented | | Sensitivity | 0.61 | 0.64 | 0.56 | 0.68 |
| qSOFA model | | Specificity | 0.66 | 0.61 | 0.73 | 0.54 |
| SOFA | 279 | — | 0.85 | 0.68 | 0.12 | 0.99 |
| Augmented SOFA | | Sensitivity | 0.85 | 0.72 | 0.12 | 0.99 |
| model | | Specificity | 0.86 | 0.69 | 0.13 | 0.99 |
| SIRS | 57 | — | 0.99 | 0.21 | 0.95 | 0.51 |
| Augmented SIRS | | Sensitivity | 0.99 | 0.27 | 0.95 | 0.64 |
| model | | Specificity | 0.99 | 0.21 | 0.95 | 0.51 |
| MEWS | 29 | — | 0.97 | 0.21 | 0.85 | 0.63 |
| Augmented | | Sensitivity | 0.98 | 0.24 | 0.85 | 0.70 |
| MEWS model | | Specificity | 0.98 | 0.21 | 0.89 | 0.63 |

The results shown in Table 6 indicate that while calculation of qSOFA is based only on a few measures and therefore is easy to estimate in the clinic, the model performance in risk stratification of patients is weak, as has been observed in several earlier studies (see M. Dorsett, M. Kroll, C. S. Smith, P. Asaro, S. Y. Liang, and H. P. Moy, “qSOFA Has Poor Sensitivity for Prehospital Identification of Severe Sepsis and Septic Shock,” Prehospital Emergency Care, vol. 21, no. 4, pp. 489-497, July 2017; A. Askim et al., “Poor performance of quick-SOFA (qSOFA) score in predicting severe sepsis and mortality—a prospective study of patients admitted with infection to the emergency department,” Scandinavian Journal of Trauma, Resuscitation and Emergency Medicine, vol. 25, no. 1, p. 56, June 2017; S. Tusgul, P.-N. Camon, B. Yersin, T. Calandra, and F. Dami, “Low sensitivity of qSOFA, SIRS criteria and sepsis definition to identify infected patients at risk of complication in the prehospital setting and at the emergency department triage,” Scandinavian Journal of Trauma, Resuscitation and Emergency Medicine, vol. 25, no. 1, p. 108, November 2017). Only about half the patients at high or low risk of developing septic shock are classified correctly. qSOFA has been observed to be a better predictor of in-hospital mortality but poorly predictive of severe sepsis(see E. P. Raith et al., “Prognostic Accuracy of the SOFA Score, SIRS Criteria, and qSOFA Score for In-Hospital Mortality Among Adults With Suspected Infection Admitted to the Intensive Care Unit,” JAMA, vol. 317, no. 3, pp. 290-300, January 2017). The results of this analysis are consistent with earlier studies showing that qSOFA score is a poor predictor of severe sepsis or septic shock. The performance of qSOFA could be improved by adding in variables from the de-novo sepsis prediction methods described herein. The SOFA score, while having good model performance in correctly classifying risk, is based on many more variables and hence is more difficult to calculate in the clinical setting. Based on the implementation of SIRS and MEWS scores, they are observed to have strong performance in identifying patients that are at low risk of septic shock but do not identify a large majority of patients at high risk of septic shock. Some previous work has shown that SIRS and SOFA scores have low sensitivity (see Tusgul et al.), validating the observations made in this example. The SOFA score would not be significantly improved by augmenting with the de-novo model while the specificity of SIRS and MEWS could be improved by augmenting with the de-novo model.

In the Examples, models were developed that identify high risk patients 24 hour prior to diagnosis of septic shock. A data-driven approach to developing the risk stratification model was adopted through use of advanced machine learning and artificial intelligence based techniques. Data-driven approaches are unbiased by current knowledge and provide an alternative to expert knowledge based, hypothesis driven approaches. Risk factors identified using advanced machine learning and artificial intelligence based methods were also used to augment currently used sepsis scores with the newly identified risk factors and improve performance of the scoring systems.

Although the present invention has been described with reference to specific example embodiments, it will be evident that various modifications and changes may be made to these embodiments without departing from the broader spirit and scope of the invention. Accordingly, the specification and drawings are to be regarded in an illustrative rather than a restrictive sense.

It will be appreciated that, for clarity purposes, the above description describes some embodiments with reference to different functional units or processors. However, it will be apparent that any suitable distribution of functionality between different functional units, processors or domains may be used without detracting from the invention. For example, functionality illustrated to be performed by separate processors or controllers may be performed by the same processor or controller. Hence, references to specific functional units are only to be seen as references to suitable means for providing the described functionality, rather than indicative of a strict logical or physical structure or organization.

Although an embodiment has been described with reference to specific example embodiments, it will be evident that various modifications and changes may be made to these embodiments without departing from the broader spirit and scope of the invention. Accordingly, the specification and drawings are to be regarded in an illustrative rather than a restrictive sense. The accompanying drawings that form a part hereof, show by way of illustration, and not of limitation, specific embodiments in which the subject matter may be practiced. The embodiments illustrated are described in sufficient detail to enable those skilled in the art to practice the teachings disclosed herein. Other embodiments may be used and derived therefrom, such that structural and logical substitutions and changes may be made without departing from the scope of this disclosure. This Detailed Description, therefore, is not to be taken in a limiting sense, and the scope of various embodiments is defined only by the appended claims, along with the full range of equivalents to which such claims are entitled.

Such embodiments of the inventive subject matter may be referred to herein, individually and/or collectively, by the term “invention” merely for convenience and without intending to voluntarily limit the scope of this application to any single invention or inventive concept if more than one is in fact disclosed. Thus, although specific embodiments have been illustrated and described herein, it should be appreciated that any arrangement calculated to achieve the same purpose may be substituted for the specific embodiments shown. This disclosure is intended to cover any and all adaptations or variations of various embodiments. Combinations of the above embodiments, and other embodiments not specifically described herein, will be apparent to those of skill in the art upon reviewing the above description.

In this document, the terms “a” or “an” are used, as is common in patent documents, to include one or more than one, independent of any other instances or usages of “at least one” or “one or more.” In this document, the term “or” is used to refer to a nonexclusive or, such that “A or B” includes “A but not B,” “B but not A,” and “A and B,” unless otherwise indicated. In the appended claims, the terms “including” and “in which” are used as the plain-English equivalents of the respective terms “comprising” and “wherein.” Also, in the following claims, the terms “including” and “comprising” are open-ended; that is, a system, device, article, or process that includes elements in addition to those listed after such a term in a claim are still deemed to fall within the scope of that claim. Moreover, in the following claims, the terms “first,” “second,” and “third” and so forth are used merely as labels, and are not intended to impose numerical requirements on their objects.

The Abstract of the Disclosure is provided to allow the reader to quickly ascertain the nature of the technical disclosure. It is submitted with the understanding that it will not be used to interpret or limit the scope or meaning of the claims. In addition, in the foregoing Detailed Description, it can be seen that various features are grouped together in a single embodiment for the purpose of streamlining the disclosure. This method of disclosure is not to be interpreted as reflecting an intention that the claimed embodiments require more features than are expressly recited in each claim. Rather, as the following claims reflect, inventive subject matter lies in less than all features of a single disclosed embodiment. Thus the following claims are hereby incorporated into the Detailed Description, with each claim standing on its own as a separate embodiment.

1. A method comprising:
accessing and/or receiving information regarding a patient, the information including an indication of whether a measured level of mean corpuscular hemoglobin for the patient fell outside a normal range for the patient, and an indication of whether a measured level of whole blood potassium for the patient fell outside a normal range for the patient;
determining, via one or more microprocessors, an estimated risk of the patient experiencing septic shock within a specified time period based on, at least, the indication of whether the measured level of mean corpuscular hemoglobin fell outside the normal range and the indication of whether the measured level of whole blood potassium fell outside the normal range; and
providing information regarding the estimated risk of the patient experiencing septic shock within the specified time period.

2. The method of claim 1, wherein the accessed and/or received information regarding the patient further comprises information regarding whether the patient has been diagnosed with at least one disease or disorder in a group of diseases and disorders; and
wherein the determining of the estimated risk of the patient experiencing septic shock within the specified time period is based on, at least, the indication of whether the measured level of mean corpuscular hemoglobin fell outside the normal range, the indication of whether the measured level of whole blood potassium fell outside the normal range, and the information regarding whether the patient has been diagnosed with at least one disease or disorder in the group.

3. (canceled)

4. The method of claim 2, wherein the determining of the estimated risk of the patient experiencing septic shock within the specified time period is based, at least in part, on a statistical model of risk using a first variable based on the indication of whether the measured level of mean corpuscular hemoglobin fell outside the normal range, a second variable based on the indication of whether the measured level of whole blood potassium fell outside the normal range, and a third variable based on whether the patient has been diagnosed with at least one disease or disorder in the group.

5.-6. (canceled)

7. The method of claim 4, wherein the model is a statistical regression model based on the first variable, the second variable, and the third variable;
wherein a value of the first variable based on the indication of whether the measured level of mean corpuscular hemoglobin fell outside the normal range is zero if the measured level fell in the normal range and is nonzero if the measured level fell outside the normal range;
wherein a value of the second variable based on the indication of whether the measured level of whole blood potassium fell outside the normal rage is zero if the measured level fell in the normal range and is nonzero if the measured level fell outside the normal range; and
wherein a value of the third variable based on whether the patient has been diagnosed with at least one disease or disorder in the group is nonzero if the patient has been diagnosed with at least one disease or disorder on the group and is zero if the patient has not be diagnosed with at least one disease or disorder on the group.

8. The method of claim 4, wherein the model is a statistical regression model based on the first variable, the second variable, and the third variable;
wherein a value of the first variable based on the indication of whether the measured level of mean corpuscular hemoglobin fell in outside normal range is 0 if the measured level fell in the normal range and is 1 if the measured level fell outside the normal range;
wherein a value of the second variable based on the indication of whether the measured level of whole blood potassium fell outside the normal range is 0 if the measured level fell in the normal range and is 1 if the measured level fell outside the normal range;
wherein a value the third variable based on whether the patient has been diagnosed with at least one disease or disorder in the group is 1 if the patient has been diagnosed with at least one disease or disorder on the group and is 0 if the patient has not be diagnosed with at least one disease or disorder on the group; and
wherein the coefficients of the statistical regression model are as follows:
intercept=0.51; first variable=−2.6; second variable=−1.9; and third variable=1.2.

9. The method of claim 4, wherein the model is a statistical regression model based on the first variable, the second variable, and the third variable;
wherein a value of the first variable based on the indication of whether the measured level of mean corpuscular hemoglobin fell outside the normal range is 0 if the measured level fell in the normal range and is 1 if the measured level fell outside the normal range;
wherein a value of the second variable based on the indication of whether the measured level of whole blood potassium fell outside the normal range is 0 if the measured level fell in the normal range and is 1 if the measured level fell outside the normal range;
wherein a value of the third variable based on whether the patient has been diagnosed with at least one disease or disorder in the group is 1 if the patient has been diagnosed with at least one disease or disorder on the group and is 0 if the patient has not be diagnosed with at least one disease or disorder on the group; and
wherein the coefficients of the statistical regression model fall in the following ranges:
0.3<intercept<0.72;
−2.90<first variable<−2.34;
−2.42<second variable<−1.31; and
0.89<third variable<1.55.

10. The method of claim 1, wherein the accessed and/or received information regarding the patient further comprises an indication of whether a measured level of blood pH for the patient fell outside a normal range for the patient; and
wherein the determining of the estimated risk of the patient experiencing septic shock within the specified time period is based, at least in part, on the indication of whether the measured level of mean corpuscular hemoglobin fell outside the normal range, the indication of whether the measured level of whole blood potassium fell outside the normal range, and the indication of whether the measured level of blood pH fell outside the normal range.

11. (canceled)

12. The method of claim 10, wherein the determining of the estimated risk of the patient experiencing septic shock within the specified time period is based, at least in part, on a statistical model of risk including variables based on whether the measured level of mean corpuscular hemoglobin fell outside the normal range, whether the measured level of whole blood potassium fell outside the normal range, and whether the measured level of blood pH fell outside the normal range.

13. The method of claim 1, wherein the accessed and/or received information regarding the patient further comprises an indication of whether a measured level of blood pH for the patient fell outside a normal range for the patient, an indication of whether mean corpuscular hemoglobin was measured, and an indication of whether whole blood potassium was measured; and
wherein the determining of the estimated risk of the patient experiencing septic shock within the specified time period is based, at least on part, on the indication of whether the measured level of mean corpuscular hemoglobin fell outside the normal range, the indication of whether the measured level of whole blood potassium fell outside the normal range, the indication of whether the measured level of blood pH fell outside the normal range, the indication of whether mean corpuscular hemoglobin was measured, and the indication of whether whole blood potassium was measured.

14. (canceled)

15. The method of claim 13, wherein the determining of the estimated risk of the patient experiencing septic shock within the specified time period is based, at least in part, on a statistical model of risk including variables based on whether the measured level of mean corpuscular hemoglobin fell outside the normal range, whether the measured level of whole blood potassium fell outside the normal range, whether the measured level of blood pH fell outside the normal range, whether mean corpuscular hemoglobin was measured, and whether whole blood potassium was measured.

16. The method of claim 1, wherein the accessed and/or received information regarding the patient further comprises an indication of whether a measured level of blood pH for the patient fell outside a normal range for the patient, an indication of whether the measured level of blood pH was measured, an indication of whether mean corpuscular hemoglobin was measured, and an indication of whether whole blood potassium was measured; and
wherein the determining of the estimated risk of the patient experiencing septic shock within the specified time period is based, at least in part, on the indication of whether the measured level of mean corpuscular hemoglobin fell outside the normal range, the indication of whether the measured level of whole blood potassium fell outside the normal range, the indication of whether the measured level of blood pH fell outside the normal range, the indication of whether the measured level of blood pH was measured, the indication of whether mean corpuscular hemoglobin was measured, and the indication of whether whole blood potassium was measured.

17. (canceled)

18. The method of claim 16, wherein the determining of the estimated risk of the patient experiencing septic shock within the specified time period is based, at least in part, on a statistical model of risk including variables based on whether the measured level of mean corpuscular hemoglobin fell outside the normal range, whether the measured level of whole blood potassium fell outside the normal range, whether the measured level of blood pH fell outside the normal range, whether mean corpuscular hemoglobin was measured, whether whole blood potassium was measured, and whether the blood pH was measured.

19. The method of claim 18, wherein a value of a first variable based on the indication of whether the measured level of mean corpuscular hemoglobin fell outside the normal range is 1 where the measured level of mean corpuscular hemoglobin fell outside the normal range and zero where the measured level of mean corpuscular hemoglobin fell within the normal range or was not measured;
wherein a value of a second variable based on the indication of whether the mean corpuscular hemoglobin was measured is 1 where the mean corpuscular hemoglobin was not measured and is 0 where the mean corpuscular hemoglobin was measured;
wherein a value of a third variable based on the indication of whether the measured level of blood pH fell outside the normal range is 1 where the measured level of blood pH fell outside the normal range and is zero where the measured level of blood pH fell within the normal range or was not measured;
wherein a value of a fourth variable based on whether the blood pH was measured is 1 where the blood pH was not measured and is 0 where the blood pH was measured; and
wherein a value of a fifth variable based on the indication of whether the measured level of whole blood potassium fell outside the normal range is 1 where the measured level of whole blood potassium fell outside the normal range and is zero where the measured level of whole blood potassium value fell within the normal range or was not measured;
wherein a value of a sixth variable based on whether the whole blood potassium was measured is 1 where the whole blood potassium was not measured and is 0 where the whole blood potassium was measured; and
wherein the coefficients of the statistical regression model fall in the following ranges:
0.38<first variable<1.08;
2.57<second variable<3.31;
1.04<third variable<1.9;
−0.57<fourth variable<−0.07;
0.81<fifth variable<2.13; and
0.77<sixth variable<1.75.

20. (canceled)

21. The method of claim 19, wherein the first variable is about 0.73, the second variable is about 2.94, the third variable is about 1.47, the fourth variable is about −0.32, the fifth variable is about 1.47, and the sixth variable is about 1.26.

22. The method of claim 21, where the intercept is zero.

23. The method of claim 1, further comprising, where the estimated risk is above a threshold value, providing an alert of a high risk of the patient experiencing septic shock within the specified time period.

24. The method of claim 23, wherein providing the alert comprises displaying the alert on a display device, transmitting the alert to one or more care providers for the patient, or both.

25. (canceled)

26. The method of claim 1, wherein the information regarding the estimated risk of the patient experiencing septic shock within the specified time period is provided to a clinical decision support system as factor in determining a treatment or care recommendation.

27. The method of claim 1, further comprising:
where the estimated risk the patient experiencing septic shock is above a threshold value, providing information to a clinical decision support system that the patient is at increased risk of septic shock.

28. The method of claim 1, wherein the method is a method of identifying a patient at increased risk of septic shock, and
wherein the method further comprises:
determining if the estimated risk is above a threshold value, and
identifying the patient as having an increased risk of septic shock where the estimated risk is above the threshold value.

29. A method of identifying a patient at increased risk of septic shock, the method comprising:
detecting a level of mean corpuscular hemoglobin in the patient's blood and identifying whether the detected level of mean corpuscular hemoglobin the patient's blood falls outside a normal range for the patient;
detecting a level of whole blood potassium for the patient and identifying whether the detected level of whole blood potassium for the patient falls outside a normal range for the patient;
detecting a level of blood pH for the patient and identifying whether the detected level of blood pH falls outside a normal range for the patient or identifying whether the patient has had a diagnosis of at least one disease or disorder in a group of diseases or disorders;
determining an estimated risk that the patient will experience septic shock within a specified time period based on the identification of whether the detected level of mean corpuscular hemoglobin falls outside the normal range, the identification of whether the detected level of whole blood potassium falls outside the normal range, and either whether patient has had a diagnosis of at least one disease or disorder from the group of diseases and disorders or the identification of whether the detected level of blood pH falls outside a normal range for the patient; and
where the estimated risk is above a threshold value, identifying the patient as having an increased risk of septic shock.

30. The method of claim 29, wherein the determining of the estimated risk of the patient experiencing septic shock within the specified time period is based, at least in part, on a statistical model of risk using a first variable based on the identification of whether the measured level of mean corpuscular hemoglobin fell outside the normal range, a second variable based on the identification of whether the measured level of whole blood potassium fell outside the normal range, and a third variable based on either whether the patient has been diagnosed with at least one disease or disorder in the group or whether the detected level of blood pH fell outside a normal range.

31.-32. (canceled)

33. The method of claim 30, wherein the model is a statistical regression model based on the first variable, the second variable, and the third variable;
wherein a value of the first variable based on the identification of whether the measured level of mean corpuscular hemoglobin fell outside the normal range is zero if the measured level fell in the normal range and is nonzero if the measured level fell outside the normal range;
wherein a value of the second variable based on the identification of whether the measured level of whole blood potassium fell outside the normal rage is zero if the measured level fell in the normal range and is nonzero if the measured level fell outside the normal range; and
wherein either a value of the third variable based on whether the patient has been diagnosed with at least one disease or disorder in the group is nonzero if the patient has been diagnosed with at least one disease or disorder on the group and is zero if the patient has not be diagnosed with at least one disease or disorder on the group, or a value of the third variable based on whether the detected level of blood pH fell outside a normal range is nonzero if the measured level fell within the normal range and is zero if the measured level fell outside the normal range.

34. The method of claim 33,
wherein the coefficients of the statistical regression model are as follows:
intercept=0.51; first variable=−2.6; second variable=−1.9; and third variable=1.2.

35. The method of claim 33, wherein the coefficients of the statistical regression model fall in the following ranges:
0.3<intercept<0.72;
−2.90<first variable<−2.34;
−2.42<second variable<−1.31; and
0.89<third variable<1.55.

36.-38. (canceled)

39. The method of claim 2, wherein the group of diseases or disorders comprises: hypersmolality, hypernatremia, acidosis, alkalosis, mixed-acid based balance disorder, fluid overload, electrolyte and fluid disorders, and angioneurotic edema; or
wherein the group of diseases or disorders comprises diseases or disorders falling in the following International Classification of Disease 9 Clinically Modified (ICD-9-CM) Codes: 2760, 2762, 2763, 2764, 2766, 27669, 2769, and 9951; or
wherein the group of diseases or disorders comprises disorders related to electrolyte balance and fluid balance.

40.-43. (canceled)

44. The method of claim 2, wherein the specified time period is less than 36 hours.

45.-46. (canceled)

47. The method of claim 1, wherein the patient is in an intensive care unit and wherein the determining of the estimated risk is specific to patients in an intensive care unit.

48. A non-transitory computer readable medium including executable instructions, that, when executed by one or more processors, perform the method of claim 1.

49. A system comprising:
a database configured to store information regarding a patient including an indication of whether a measured level of mean corpuscular hemoglobin for the patient fell outside a normal range for the patient, an indication of whether a measured level of whole blood potassium for the patient fell outside a normal range for the patient, and either information regarding current and prior diagnoses of diseases and disorders for the patient or at least one of an indication of whether a measured level of blood pH for the patient fell outside a normal range and an indication that blood pH has not been measured for the patient; and
a septic shock risk assessment module configured to:
receive or access the information regarding the patient from the database;
determine whether the patient has been diagnosed with one or more diseases and disorders in a group of diseases and disorders; and
determine an estimated risk of the patient experiencing septic shock within a specified time period based on, at least, the indication of whether the measured level of mean corpuscular hemoglobin fell outside the normal range, the indication of whether the measured level of whole blood potassium fell outside the normal range, and either the determination of whether the patient has been diagnosed with the at least one disease or disorder in the group of diseases and disorders or at least one of the determination of whether the measured level of blood pH feel outside the normal range and the indication that the level of blood pH has not been measured.

50.-51. (canceled)

52. The system of claim 49, wherein the risk assessment module is further configured to determine whether the estimated risk is larger than a threshold value.

53. The system of claim 49, further comprising an alert module configured to provide or transmit an alert when the estimated risk for the patient is larger than the threshold value.

54. The system of claim 49, wherein the system is a clinical decision support system and wherein information regarding the estimated risk of septic shock is used to determining a treatment or a care recommendation for the patient.
